(19) United States
(12) Patent Application Publication
(10) Pub. No.: US 2007/0264653 A1
(43) Pub. Date: Nov. 15, 2007

(54) Method of identifying a biological sample for methylation analysis

(76) Inventors: Kurt Berlin, Stahnsdorf (DE); Dimo Dietrich, Berlin (DE); Philipp Schatz, Berlin (DE); Michael Wandell, Mercer Island, WA (US); Antje Kluth, Wentorf (DE); Reimo Tetzner, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 11/716,207

(22) Filed: Mar. 10, 2007

(30) Foreign Application Priority Data
Mar. 10, 2006 (EP) 06090032.1
May 30, 2006 (EP) 06090091.7

(51) Int. Cl. C12Q 1/68

(52) U.S. Cl. 435/6

(57) ABSTRACT

Aspects of the present invention relate to compositions and methods of identifying at least one biological sample in the field of methylation analysis. In particular aspects at least one biological sample is provided, at least one identifier is applied for each sample, the applied identifier(s) are detected or quantified, and the methylation analysis is performed. Additional aspects provide a methods for testing an experimental procedure. Additional aspects provide kits suitable for realizing the aspects of the invention.

### FIELD OF THE INVENTION

The invention relates generally to novel and substantially improved compositions and methods of identifying a biological sample for methylation analysis comprising identifier, in particular their application, use and detection.

### BACKGROUND OF ASPECTS OF THE INVENTION

Methylation analysis. Many diseases, in particular cancer diseases, are accompanied by a modified gene expression. This may be a mutation of the genes themselves, which leads to an expression of modified proteins or to an inhibition or over-expression of the proteins or enzymes. A modulation of the expression may however also occur by epigenetic modifications, in particular DNA methylation. Such epigenetic modifications do not affect the actual DNA coding sequence. It has been found that DNA methylation processes have substantial implications for the health, and it seems to be clear that knowledge about methylation processes and modifications of the methyl metabolism and DNA methylation are essential for understanding diseases, for the prophylaxis, diagnosis and therapy of diseases.

The precise control of genes, which represent a small part only of the complete genome of mammals, is a question of the regulation under consideration of the fact that the main part of the DNA in the genome is not coding. The presence of such trunk DNA containing introns, repetitive elements and potentially actively transposable elements, requires effective mechanisms for their durable suppression (silencing). Apparently, the methylation of cytosine by S-adenosylmethionine (SAM) dependent DNA methyltransferases, which form 5-methylcytosine, represents such a mechanism for the modification of DNA-protein interactions. Genes can be transcribed by methylation-free promoters, even when adjacent transcribed or not-transcribed regions are widely methylated. This permits the use and regulation of promoters of functional genes, whereas the trunk DNA including the transposable elements is suppressed. Methylation also takes place for the long-term suppression of X-linked genes and may lead to either a reduction or an increase of the degree of transcription, depending on where the methylation in the transcription unit occurs.

Nearly the complete natural DNA methylation in mammals is restricted to cytosine-guanosine (CpG) dinucleotide palindrome sequences, which are controlled by DNA methyl transferases. CpG dinucleotides are about 1 to 2% of all dinucleotides and are concentrated in so-called CpG islands. A generally accepted definition of CpG islands means that a 200 bp long DNA region has a CpG content of at least 50%, and that the ratio of the number of observed CG dinucleotides and the number of the expected CG dinucleotides is larger than 0.6 (Gardiner-Garden, M., Frommer, M. (1987) J. Mol. Biol. 196, 261-282; this cited reference is incorporated by reference to its entirety). Typically, CpG islands have at least 4 CG dinucleotides in a sequence having a length of 100 base pairs.

If CpG islands are present in promoter areas, they have often a regulatory function for the expression of the respective gene. If the CpG island is hypomethylated, expression can take place. Hypermethylation often leads to the suppression of the expression. In the normal state, a tumor suppressor gene is hypomethylated. If a hypermethylation takes place, this will lead to a suppression of the expression of the tumor suppressor gene, which is frequently observed in cancer tissues. In contrast thereto, oncogenes are hypermethylated in healthy tissue, whereas in cancer tissue they are frequently hypomethylated.

By the methylation of cytosine, regularly the binding of proteins regulating the transcription is prevented. This leads to a modification of the gene expression. With regard to cancer, for instance the expression of cell division regulating genes is affected thereby, i.e. for instance the expression of apoptosis genes is regulated down, whereas the expression of oncogenes is regulated up. The hypermethylation of the DNA has however also a long-term influence on the regulation. By the methylation of cytosine, histone de-acetylation proteins can bind by their 5-methylcytosine-specific domain to the DNA. This has as a consequence that histones are de-acetylated, which will lead to a tighter compacting of the DNA. Thereby, regulatory proteins do not have the possibility anymore to bind to the DNA.

For the reason of this, the detection of DNA methylation is important with respect to diagnosing a disease, prognosing a disease, predicting a treatment response, diagnosing a predisposition for a disease, diagnosing a progression of a disease, grading a disease, staging a disease, classifying a disease, characterizing a disease, or for identifying a new marker associated with a disease. An overview of method for DNA methylation analysis can be gathered from Laird P W. “The power and the promise of DNA methylation markers” Nat Rev Cancer April 2003;3(4):253-66. Many methods for methylation analysis are based on treatment of genomic DNA with reagent that differentiates between methylated and unmethylated cytosines. In many cases this reagent is a bisulfite reagent which leads to a conversion of unmethylated cytosines to uracil or after amplification to thymin while methylated cytosines remain unchanged.

Pronounced need in the art. Like many modern laboratory workflows methods for methylation analysis are characterized in that a large number of samples has to be processed. Thereby it is irrelevant, if the methods or workflows are applied for diagnosing a disease, prognosing a disease, predicting a treatment response, diagnosing a predisposition for a disease, diagnosing a progression of a disease, grading a disease, staging a disease, classifying a disease, characterizing a disease, or for identifying a new marker like methylation, RNA, or protein which is associated with a disease.

In any case it is import that samples are not interchanged and no sample is contaminated with another sample. As relevant prior art the following is considered:

According to WO9943855 endogeneous polymorphic sequences are used as unique identifier for biological samples. These identifiers link the sample to its source and other relevant information.

According to U.S. Pat. No. 6,153,389 biological forensic or medical samples are marked by the addition of nucleic acids of known sequence. It further utilizes primers and their use for the detection of the added nucleic acid in an amplification reaction resulting in a nucleic acid molecule of specific length.

Currently the applicant is not aware of any prior art, which addresses the question of detecting sample interchange and/or sample crosscontamination for methylation analysis. The two above cited documents does not teach a method for labelling a biological sample which survives a bisulfite treatment as it is used many times in methylation analysis.

### SUMMARY OF ASPECTS OF THE INVENTION

Aspects of the present invention relate to compositions and methods of identifying a biological sample in the field of methylation analysis comprising at least one identifier, in particular their application, use and detection.

Particular aspects provide compositions and methods of identifying a biological sample in the field of methylation analysis, wherein biological samples are provided, one or more identifiers are applied to one sample, the applied identifier(s) are detected or quantified, and the DNA methylation of each biological sample is analyzed.

Particular aspects provide compositions and methods of identifying a biological sample in the field of methylation analysis, wherein the DNA of the biological sample including the identifier is brought into contact with a reagent or enzyme which differentiates between methylated or unmethylated cytosine positions.

Particular aspects provide compositions and methods of identifying a biological sample in the field of methylation analysis, wherein the biological sample and the identifier are subjected to experimental procedures. For example, but not limited to, wherein the DNA of the sample is isolated, bisulfite treated, purified, and desulfonated.

Particular aspects provide compositions and methods of identifying a biological sample in the field of methylation analysis, wherein the detection or quantification is realized simultaneously with the methylation analysis.

Particular aspects provide compositions and methods of identifying a biological sample in the field of methylation analysis, wherein the identifier is at least in parts a nucleic acid. In particular aspects the identifier comprises at least one cytosine, at least one guanin, or both; is a variant of a polymorphism; and/or has a similar base composition as the DNA section of interest. In particular aspects the identifier is part of the endogeneous genomic DNA of the biological sample. In other particular aspects the identifier is part of a external DNA molecule which is added to the biological sample shortly after collecting the sample or at the beginning of an experimental procedure.

Particular aspects provide compositions and methods of identifying a biological sample in the field of methylation analysis, wherein the applied identifiers of different samples are assigned to different set of identifiers according to their biological, chemical, or physical properties. In particular aspects the identifiers are assigned to sets of sequence polymorphic variants, to sets of length polymorphic variants, to sets of single nucleotide polymorphic variants, or to sets of deletion polymorphic variants.

Particular aspects provide compositions and methods of identifying a biological sample in the field of methylation analysis, wherein the identifier is detected or quantified by nucleic acids or analogs thereof which comprise at least one cytosine and/or at least one guanin.

Particular aspects provide compositions and methods of identifying a biological sample in the field of methylation analysis, which can be used as a method of detection of sample interchange, crosscontamination, or both; as a method of identifying a sample in a pooled sample set; as a method of an amplification inhibition; as a method of normalization, calibration or both; as a method of identification of a carry over contamination or both; as a method of determining the rate of DNA conversion; or as a method for assessing the success of a hybridization step.

Particular aspects provide compositions and methods for controlling the correctness of a process or method.

Particular aspects provide also kits for realizing said particular aspects of the invention.

### BRIEF DESCRIPTION OF THE FIGURES

FIG. 1 shows an overview over one embodiment of the invention. According to this embodiment two identifiers are assigned to each other, whereby each identifier belongs to a different set of identifiers. One set contains 8 variants of a sequence polymorphism, and the other 8 variants of a length polymorphism. The primers for detection are the same for every variant of a set.

FIG. 2 shows exemplary an overview over a set of 24 samples. A unique combination of identifiers is applied to each sample. Each identifier is detected by means of hybridization of the correspondent amplification products. As can be easily seen, every identifier combination hence every sample has its own characteristic hybridization pattern.

FIG. 3 shows at the top exemplary how a sample interchange of sample 2 with sample 17 is detected. FIG. 3 shows in the lower part exemplary how a sample cross contamination of sample 2 with sample 17 is detected.

FIG. 4 shows schematic drawings of a hybridization. In A the probe orientation at the array is shown. B and C show hybridizations of samples. In B two plasmids are used which were generated by domain-primer 1+domain-primer 2 and domain-primer 1+domain-primer 3. In C two plasmids are used which were generated by domain-primer 1+domain-primer 2 and domain-primer 1+domain-primer 4.

FIG. 5 shows a schematic drawing of a hybridized microarray detecting a contamination of the sample. A combination of two plasmids was used.

FIG. 6A shows an agarose gel analysis of amplificates of the linearized bisulfite treated plasmid 23. 100 pg of bisulfite treated linearized plasmid 23 were used for amplification.

FIG. 6B) shows an agarose gel analysis of amplificates of the linearized bisulfite treated plasmid 195. 100 pg of bisulfite treated linearized plasmid 23 were used for amplification. (m=size standard, bright band is about 200 bp)

FIG. 7 shows the image of two hybridized array tubes. A) Amplificates derived from molecular identification plasmid 23 were used for hybridization. B) Amplificates derived from molecular identification plasmid 195 were used for hybridization. The amplificates of each of the two molecular identification plasmids hybridizes specifically two oligonucleotides of the array tube (dark spots marked by quadrats). Dark spots at the corner of the image show control spots necessary for scanning the array tubes. Light grey spots represent unspecific hybridization.

FIG. 8A shows an schematic overview of a identification pattern according to the invention. A first identifier is assigned to samples of the columns 1, 5, 9 of a microtiter plate. A second identifier is assigned to samples of the columns 2, 6, 10. A third identifier is assigned to samples of the column 3, 7, 11. (Identifier X=light grey, identifier Y=middle grey, identifier Z=dark grey).

FIG. 8B shows an schematic overview of the determined identifier pattern by analysis. Every detected identifier identity is assigned to the position of the sample from which an aliquot was used for determining the identifier identity. It is obvious that a sample interchange occurred in the first run (samples 5 and 6) as well as in the third run (samples 12 and 13).

### DETAILED DESCRIPTION OF ASPECTS OF THE INVENTION

For achieving various technical objects, aspects the invention teach compositions and methods of identifying a biological sample for methylation analysis comprising identifier, in particular their application, use and detection. Said compositions and methods comprise providing at least one biological sample, applying at least one identifier, detecting or quantifying the applied identifier(s) and performing a methylation analysis.

Particular aspects provide methods comprising at least one identifier and at least one nucleic acid, which enables a detection or quantification of the at least one identifier even after the treatment with bisulfite sulfite or any other correspondent DNA converting reagent. Particular aspects provide further methods for simultaneous detection or quantification of the applied at least one identifier and the detection or quantification of the methylation of the genomic DNA of the provided biological sample. Particular aspects provide suitable combinations and adjustments of these methods with each other in a manner that actually meets the technical object(s).

### ADVANTAGES OF ASPECTS OF THE INVENTION

In particular aspects, the exemplary inventive method has the advantage that it enables the use of identifiers in conjunction with bisulfite conversion of DNA. In particular, it therefore has the following advantages for methylation analysis:
It enables a detection of sample interchange.
It enables a detection of sample crosscontamination.
It enables a detection of carry over contamination.
It enables further a normalization and/or calibration of the sample or of the used methylation analysis method.
It enables a identification of a sample in a pooled set of samples.
It enables to calculate a conversion rate for bisulfite treatment.
It enables a detection of inhibition of subsequent processes in particular PCR based analysis.
It enables an assessment of the success of a hybridization step.

In particular aspects, the exemplary inventive method has the advantage that it controlls the error free and accurate run of a process or method, in particular of a high throughput method. Thereby said method can be a method for diagnosis, prognosis, or for marker discovery.

### METHOD OF ASPECTS OF THE INVENTION

The method of the invention is a method for labelling or marking a biological sample in particular in the field of methylation analysis. The method comprises the following in arbitrary order:

A) The providing of a biological sample which comprises differentially methylated DNA.

B) The assignment of one or more identifiers, preferably nucleic acids or at least in parts nucleic acids.

C) The execution of an experimental procedure, which analyses the methylation of the provided DNA, the assigned at least one identifier, or both. Preferably this workflow enables detection or quantification of the methylation, the identifier, or both. In particular the detection or quantification of the methylation and the identifier are realized simultaneously.

In brief, in particular aspects, the method of the invention is a method of identifying at least one biological sample in the field of methylation analysis, comprising in arbitrary order:
providing a sample set of at least one biological sample, wherein at least one sample comprises genomic DNA differentially methylated at least at one position;
applying at least one identifier for each sample, wherein the applied at least one identifier does not interfere with subsequent analysis;
subjecting each sample to a detection or quantification reaction specific for the one or more applied identifiers; and
subjecting each sample to methylation analysis.

In particular aspects, the method of the invention is a method of identifying at least one biological sample in the field of methylation analysis, wherein the identifier is part of the genomic DNA of the biological sample.

In particular aspects, the method of the invention is a method of identifying at least one biological sample in the field of methylation analysis, wherein the identifier is added to the biological sample.

According to an embodiment, the method of the invention is a method of identifying at least one biological sample in the field of methylation analysis, comprising
providing a sample set of at least one biological sample, wherein at least one sample comprises genomic DNA differentially methylated at least at one position;
applying at least one identifier for each sample, wherein the applied at least one identifier does not interfere with subsequent analysis;
subjecting each sample to a detection or quantification reaction specific for the one or more applied identifiers; and
subjecting each sample to methylation analysis.

Thereby the different steps can be performed in arbitrary order. In a preferred embodiment the identifier is a nucleic acid which is part of a sample endogeneous DNA molecule, in particular a genomic DNA molecule. In another preferred embodiment, the identifier is a nucleic acid which is added to a sample. In a preferred embodiment, the identifier is detected or quantified before methylation analysis of the provided DNA. In another preferred embodiment, the identifier is detected or quantified subsequent to methylation analysis of the provided DNA. In a particular preferred embodiment, the detection or quantification of the identifier is carried out simultaneously with the methylation analysis.

A preferred embodiment further comprises at least one of the following
contacting the DNA of each sample with a reagent or enzyme which differentiates between a methylated or an unmethylated position;
processing the sample set according to an experimental procedure.

In a preferred embodiment, the identifier can be detected or quantified in between or subsequent to an experimental procedure. For example, but not limited to it, such an experimental procedure can, comprise a procedure for isolating genomic DNA, a procedure for treating genomic DNA with a reagent which differentiates between methylated and unmethylated DNA, a procedure for purifying DNA, and/or a procedure for detecting or quantifying DNA methylation. Suitable experimental procedures are for example but not limited to described in PCT/US06/14667 or in PCT/US05/35317 (these references are herewith incorporated by reference to their entirety).

A particular preferred embodiment, comprises
providing a sample set of at least one biological sample, wherein at least one sample comprises genomic DNA differentially methylated at least at one position,
applying at least one identifier for each sample,
contacting the DNA of each sample with a reagent or enzyme which differentiates between a methylated or an unmethylated position,
detecting or quantifying the applied one or more identifiers for each sample,
subjecting each sample to methylation analysis.

According to a particular preferred embodiment, the detecting or the quantifying of the applied one or more identifiers for each sample and the methylation analysis of each sample are realized simultaneously.

According to a preferred embodiment, the identifier is at least in parts part of a larger molecule; part of an endogeneous molecule of the respective sample; part of an exogenous molecule added to the respective sample; a section of genomic DNA or total genomic DNA derived from a plant; a section of genomic DNA or total genomic DNA derived from a bacteria; a section of genomic DNA or total genomic DNA derived from a non-vertebrate; a section of genomic DNA or total genomic DNA derived from a vertebrate; a short tandem repeat a variant of a deletion polymorphism; a variant of a single nucleotide polymorphism; a variant of a length polymorphism; an artificial nucleic acid; a circular nucleic acid; a circular DNA; a plasmid; a polynucleotide; an oligonucleotide; a PNA; a PNA-oligomer; a PNA-polymer; a LNA; a LNA-oligomer; a LNA-polymer; a RNA; a RNA-oligomer; a RNA-polymer; an artificial methylation; or combinations thereof.

According to a preferred embodiment, different identifiers are assigned to different sets of identifiers according to their respective biological, chemical, or physical properties.

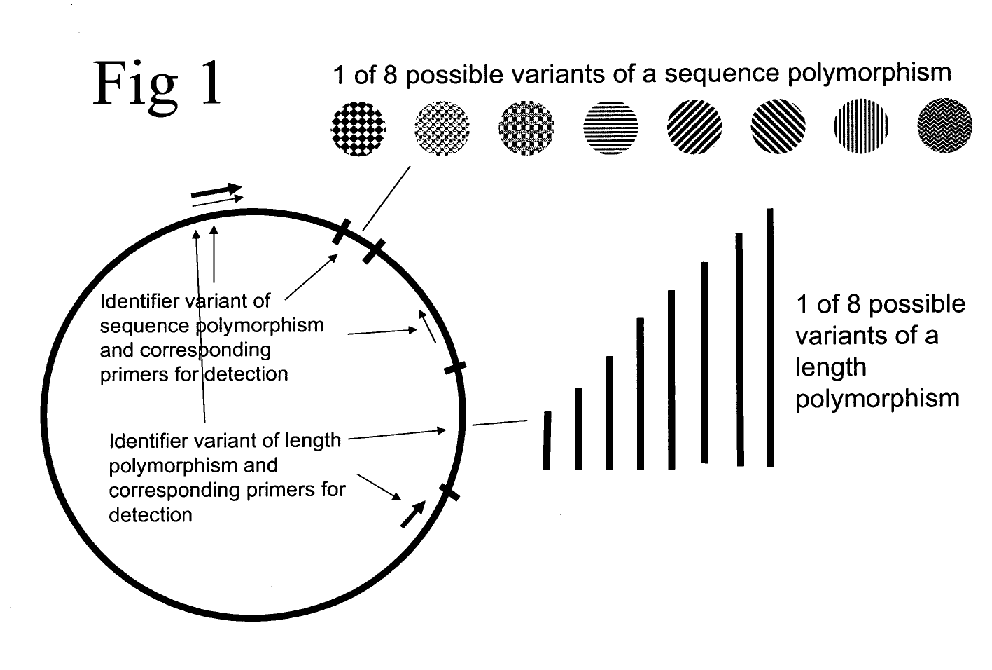

A preferred embodiment is characterized in that a representative of each of at least two sets of identifiers is comprised in a plasmid. Such a plasmid can be for example, but not limited to, a plasmid as shown in FIG. 1. A plasmid according to FIG. 1 comprises a variant of a sequence polymorphism out of 8 variants and a variant of a length polymorphism out of 8 variants. Thereby each variant of the sequence polymorphism and each variant of the length polymorphism represents an identifier. Furthermore, all variants of the sequence polymorphism represent a set of identifiers and all variants of the length polymorphism represent second set of identifiers. This combination of different sets of identifiers has the advantage that different samples can be identified according to different methods. A simple identification of corresponding samples is for example possible by detection of the different variants of length polymorphism by means of PCR and gel electrophoresis. For some applications, it might be favourable to identify samples according to the sequence polymorphic variants. For example, but not limited to, in case the methylation analysis of the DNA of biological sample comprises PCR and detection of the PCR products by hybridization. In this case it is easily possible also to perform an amplification of the different variants of the sequence polymorphism and detect them also by hybridization. Preferably the methylation analysis and the detection of the identifier are performed simultaneously. If desired it is also possible to quantify the identifier. This allows for example, but not limited to, drawbacks onto the amount of crosscontamination of the original sample.

According to a preferred embodiment, the first set of identifiers comprises a sequence polymorphism and wherein the second set of identifiers comprises a length polymorphism.

According to a preferred embodiment, the identifier is a nucleic acid and additionally
forms no stable secondary structure;
comprises at least one oligonucleotide binding site covering converted cytosine positions;
is characterized by a similar base composition as the analyzed genomic DNA of the provided sample;
is a polymorphic sequence of about 5, about 10, about 15, about 20, about 25, about 30, about 35, about 40, about 50, about 75, about 100, or about 200 nucleotides;
has a content of cytosin-nucleotides and guanosin-nucleotides of about 15%, about 20%, about 30%, about 40%, about 50%, about 60%, about 70%, or about 80%; or
combinations thereof. Preferably, an identifier comprises also additionally one or more CpG dinucleotide.

According to a preferred embodiment, the identifier is a nucleic acid and additionally
forms no stable secondary structure;
comprises at least one oligonucleotide binding site covering converted cytosine positions;
is characterized by a similar base composition as the analyzed genomic DNA of the provided sample;
is a polymorphic sequence of about 5, about 10, about 15, about 20, about 25, about 30, about 35, about 40, about 50, about 75, about 100, or about 200 nucleotides; and
has a content of cytosin-nucleotides and guanosin-nucleotides of about 15%, about 20%, about 30%, about 40%, about 50%, about 60%, about 70%, or about 80%. Preferably, an identifier comprises also additionally one or more CpG dinucleotide.

According to a preferred embodiment, the identifier is a nucleic acid and additionally
forms no stable secondary structure;
comprises at least one cytosine-free, guanin-free or cytosine-free and guanin-free oligonucleotide binding site;
is characterized by a similar base composition as the analyzed genomic DNA of the provided sample;
is a polymorphic sequence of about 5, about 10, about 15, about 20, about 25, about 30, about 35, about 40, about 50, about 75, about 100, or about 200 nucleotides;
has a content of cytosin-nucleotides and guanosin-nucleotides of about 15%, about 20%, about 30%, about 40%, about 50%, about 60%, about 70%, or about 80%; or
combinations thereof. Preferably, an identifier comprises also additionally no CpG dinucleotide.

According to a preferred embodiment, the identifier is a nucleic acid and additionally
forms no stable secondary structure;
comprises at least one cytosine-free, guanin-free or cytosine-free and guanin-free oligonucleotide binding site;
is characterized by a similar base composition as the analyzed genomic DNA of the provided sample;
is a polymorphic sequence of about 5, about 10, about 15, about 20, about 25, about 30, about 35, about 40, about 50, about 75, about 100, or about 200 nucleotides; and
has a content of cytosin-nucleotides and guanosin-nucleotides of about 15%, about 20%, about 30%, about 40%, about 50%, about 60%, about 70%, or about 80%. Preferably, an identifier comprises also additionally no CpG dinucleotide.

According to a preferred embodiment, the identifier is a variant of a sequence polymorphism and additionally
comprises about 1, about 10, about 20, about 30, about 40, about 50, about 60, about 70, about 80, about 90, or about 100 variable nucleotide sites;
forms no stable secondary structure; or both. Preferably, such an identifier comprises also additionally a part of about 1, about 10, about 20, about 30, about 40, about 50, about 60, about 70, about 80, about 90, about 100 nucleotides which are free of cytosine, guanine or both.

According to a preferred embodiment, the identifier is a variant of a sequence polymorphism and additionally
comprises about 1, about 10, about 20, about 30, about 40, about 50, about 60, about 70, about 80, about 90, or about 100 variable nucleotide sites;
forms no stable secondary structure; and
comprises a part of about 1, about 10, about 20, about 30, about 40, about 50, about 60, about 70, about 80, about 90, about 100 nucleotides which are free of cytosine, guanine or both.

In a preferred embodiment, the identifier is a variant of a sequence polymorphism and additionally
comprises about 5, about 10, about 15, about 20, or about 25 variable nucleotide sites;
forms no stable secondary structure;
has a content of cytosin-nucleotides and guanosin-nucleotides of about 20%, about 30%, about 40%, about 50%, about 60%, about 70%, or about 80%; or
combinations thereof. Preferably, such an identifier comprises also additionally a part of about 5, about 10, about 15, about 20, about 25, or about 30 nucleotides which are free of cytosine, guanine, or both.

Embodiments comprising an identifier with a cytosine-free region, a guanin-free region, or both are particular preferred in case of a conversion-specific detection. A person skilled in the art knows numerous suitable detection methods for example but not limited to real time based PCR methods comprising the use of conversion-specific primers, probes and/or blockers.

In a preferred embodiment, the identifier is a variant of a sequence polymorphism and additionally
comprises about 5, about 10, about 15, about 20, or about 25 variable nucleotide sites;
forms no stable secondary structure;
comprises a part of about 5, about 10, about 15, about 20, about 25, or about 30 nucleotides which are free of cytosine, guanine or both; and
has a content of cytosin-nucleotides and guanosin-nucleotides of about 20%, about 30%, about 40%, about 50%, about 60%, about 70%, or about 80%.

In a preferred embodiment, the identifier is a variant of a length polymorphism and additionally
has a length difference of about 10, about 100, about 200, about 300, about 400, about 500, about 600, about 700, about 800, about 900, or about 1000 nucleotides compared to other used nucleic acid length polymorphic identifiers;
is of about 10, about 100, about 500, about 1,000, about 1,500, about 2,000, about 2,500, about 3,000, about 3,500, about 4,000, about 4,500, about 5,000, about 5,500, about 6,.000, about 6,500, about 7,000, about 7,500, about 8,000, about 8,500, about 9,000, about 9,500, or about 10,000 nucleotides in length;
is derived from non-human DNA; or
combinations thereof.

In a preferred embodiment, the identifier is a variant of a length polymorphism and additionally
has a length difference of about 10, about 100, about 200, about 300, about 400, about 500, about 600, about 700, about 800, about 900, or about 1000 nucleotides compared to other used nucleic acid length polymorphic identifiers; and
is of about 10, about 100, about 500, about 1.000, about 1,500, about 2,000, about 2,500, about 3,000, about 3,500, about 4,000, about 4,500, about 5,000, about 5,500, about 6,000, about 6,500, about 7.000, about 7.500, about 8.000, about 8.500, about 9.000, about 9.500, or about 10,000 nucleotides in length;
is derived from non-human DNA.

In a preferred embodiment, the identifier is a variant of a length polymorphism and additionally
is either of about 5, about 25, about 50, about 75, about 100, about 125, about 150, about 175, about 200, about 225, about 250, about 275, about 300, about 325, about 350, about 375, about 400, about 425, about 450, about 475, or about 500 nucleotides in length;
is derived from non-human DNA;

has a content of cytosin-nucleotides and guanosin-nucleotides of about 20%, about 30%, about 40%, about 50%, about 60%, about 70%, or about 80%; or
combinations thereof.

In a preferred embodiment, the identifier is a variant of a length polymorphism and additionally
is either of about 5, about 25, about 50, about 75, about 100, about 125, about 150, about 175, about 200, about 225, about 250, about 275, about 300, about 325, about 350, about 375, about 400, about 425, about 450, about 475, or about 500 nucleotides in length;
is derived from non-human DNA; and
has a content of cytosin-nucleotides and guanosin-nucleotides of about 20%, about 30%, about 40%, about 50%, about 60%, about 70%, or about 80%.

In a preferred embodiment, the identifier comprises a tag selected from the group comprising dye, fluorescent dye, chemiluminescent dye, Cy5, Cy3, TAMRA, FAM, intercalating dye, ethidiumbromid, SYBR Green, PicoGreen, TOTO, BEBO, BETO, BOXTO, BO, BO-PRO, TO-PRO, YO-PRO, green fluorescent protein (all various colors), tag, epitope tag, peptide, polypeptide, protein, natural amino acids, non-natural amino acids, D-amino acids, norleucine, ethionine, canavanine, perthiaproline, 3,4-dehydroproline, azetidine-2-carboxylic acid, selenomethionine, aminohexanoic acid, telluromethionine, homoallylglycine, homopropargylglycine, 2-butynylglycine, azidohomolanine, homoproparglycine, sacccharide, hormon, lipid, mass label, particle, gold particle, silver particle, platin particle, polystyrol particle, polypropylen particle, or combinations thereof. In a preferred embodiment, the identifier is embedded into paraffin as a paraffin embedded code. Preferably, the identifier is embedded into paraffin simultaneously with the biological sample or after the embedment of the biological sample. Preferably, an unembedded identifier is added to embedded biological sample

According to a preferred embodiment, the reagent that differentiates between a methylated or an unmethylated position is an enzyme, preferably a nuclease. According to this embodiment the one or more identifiers comprise one or more recognition sites for the used enzyme and can be used as a control for the enzymatic activity. For example but not limited to it, said enzyme is an endonuclease whose recognition site comprises a CpG dinucleotide and cuts DNA only in case it is methylated or in case it is unmethylated. A person skilled in the art is aware of a suitable enzyme. For example, but not limited to it, McrBC, Bisl, or Glal in case the recognition site is methylated or for example, but not limited to it, BstUl, Bshl236l, Accll, BstFNI, Mvnl, Hpall (Hapll), Hhal, Acil, Smal, HinP1l, or HpyCH4IV in case the recognition site is unmethylated. Of course, a mixture of two or more of such enzymes is also preferred.

In a preferred embodiment, the reagent that differentiates between a methylated or an unmethylated position is a bisulfite reagent, wherein the methylated or unmethylated position is a cytosine position, or both.

In a preferred embodiment, the experimental procedure comprises one or more of the following sample pooling, DNA isolation, DNA pooling, DNA concentration, DNA purification, bisulfite treatment, desulfonation, amplification.

In a preferred embodiment these steps are essentially carried out as described in PCT/US06/14667 or in PCT/US05/35317 (these references are herewith incorporated by reference to their entirety).

According to a preferred embodiment, a bisulfite treatment is essentially carried out as described in WO05/038051 (this reference is incorporated by its entirety). According to this, in one embodiment DNA is reacted with a bisulfite reagent, characterized in that said reaction is carried out in the presence of a compound out of the group of dioxane, one of its derivatives and a similar aliphatic cyclic ether.

In an embodiment DNA is reacted with a bisulfite reagent, characterized in that said reaction is carried out in the presence of a compound of the following formula:

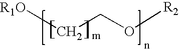

n=1-35000

m=1-3

R1=H, Me, Et, Pr, Bu

R2=H, Me, Et, Pr, Bu

Preferred are thus n-alkylene glycol compounds, particularly their dialkyl ethers, and especially diethylene glycol dimethyl ether (DME).

The bisulfite conversion may take place both in solution as well as also on DNA bound to a solid phase. Preferably sodium disulfite (=sodium bisulfite/sodium metabisulfite) is used, since it is more soluble in water than sodium sulfite. The disulfite salt disproportionates in aqueous solution to the hydrogen sulfite anions necessary for the cytosine conversion. When bisulfite concentration is discussed below, this refers to the concentration of hydrogen sulfite and sulfite anions in the reaction solution. For the method according to the invention, concentration ranges of 0.1 to 6 mol/l are possible. Particularly preferred is a concentration range of 1 to 6 mol/l, and most particularly preferred, 2-4 mol/l. However, when dioxane is used, the maximal concentration of bisulfite that can be used is smaller (see below). In selecting the bisulfite concentration, one must consider that a high concentration of bisulfite leads to a high conversion, but also leads to a high decomposition rate due to the lower pH.

Dioxane can be utilized in different concentrations. Preferably, the dioxane concentration amounts to 10 to 35% (vol/vol), particularly preferred is 20 to 30%, and most particularly preferred is 22 to 28%, especially 25%. A dioxane concentration higher than 35% is problematic, since this results in a formation of two phases within the reaction solution. In the particularly preferred embodiments with a dioxane concentration of 22-28%, the final preferred bisulfite concentration amounts to 3,3 to 3,6 mol/l, and in the most particularly preferred embodiment with a dioxane concentration of 25%, it amounts to 3,5 mol/l (see Examples).

The n-alkylene glycol compounds according to the invention can be utilized in a different concentration range. DME is preferably used in concentrations between 1-35% (vol/vol). There is preferably between 5 and 25%, and most preferably 10% DME.

The preferred scavengers utilized according to the invention are chromane derivatives, e.g., 6-hydroxy-2,5,7,8,-tetramethylchromane 2-carboxylic acid (also known as: Trolox-C™) or trihydroxybenzoe acid and derivates thereof, e.g. Gallic acid (see: PCT/EP2004/011715 which is incorporated by reference in its entirety). Further scavengers are listed in the patent application WO 01/98528 (=DE 100 29 915;=U.S. application Ser. No. 10/311,661; incorporated herein in its entirety).

The bisulfite conversion can be conducted in a wide temperature range from 0 to 95° C. However, as at higher temperatures the rates of both the conversion and decomposition of the DNA increase, in a preferred embodiment the reaction temperature lies between 0-80° C., preferably between 30-80° C. Particularly preferred is a range between 50-70° C.; most particularly preferred between 57-65° C.

The optimal reaction time of the bisulfite treatment depends on the reaction temperature. The reaction time normally amounts to between 1 and 18 hours (see: Grunau et al. 2001, Nucleic Acids Res. 2001, 29(13):E65-5; incorporated by reference herein in its entirety). The reaction time is ordinarily 4-6 hours for a reaction temperature of 60° C.

In a particularly preferred embodiment of the method according to the invention, the bisulfite conversion is conducted at mild reaction temperatures, wherein the reaction temperature is then clearly increased for a short time at least once during the course of the conversion. In this way, the effectiveness of the bisulfite conversion can be surprisingly clearly increased. The temperature increases of short duration are named “thermospikes” below. The “standard” reaction temperature outside the thermospikes is denoted as the basic reaction temperature. The basic reaction temperature amounts to between 0 and 80° C., preferably between 30-80° C., more preferably between 50-70° C., most preferably between 57-65° C., as described above.

The reaction temperature during a thermospike is increased to over 85° C. by at least one thermospike. The optimal number of thermospikes is a function of the basic reaction temperature. The higher the optimal number of thermospikes is, the lower is the basic reaction temperature. At least one thermospike is necessary in each case. And, on the other hand, in principle, any number of thermospikes is conceivable. Of course, it must be considered that with a large number of temperature increases, the decomposition rate of the DNA also increases, and an optimal conversion is no longer assured. The preferred number of thermospikes is thus between 1 and 10 thermospikes each time, depending on the basic reaction temperature. A number of two to 5 thermospikes is thus particularly preferred. The thermospikes increase the reaction temperature preferably to 85 to 100° C., particularly preferably to 90-100° C., and most preferably to 94° C-100° C.

The duration in time of the thermospikes also depends on the volume of the reaction batch. It must be assured that the temperature is increased uniformly throughout the total reaction solution. For a 20 μl reaction batch when using a thermocycler a duration between 15 seconds and 1.5 minutes, especially a duration between 20 and 50 seconds is preferred. In a particular preferred embodiment the duration is 30 seconds. Operating on a volume of 100 μl the preferred range lies between 30 seconds and 5 minutes, especially between 1 and 3 minutes. Particularly preferred are 1.5-3 minutes. For a volume of 600 μl, a duration of 1 to 6 minutes, is preferred, especially between 2 and 4 minutes. Particularly preferred is a duration of 3 minutes. A person skilled in the art will easily be able to determine suitable durations of thermospikes in relation to a variety of reaction volumes. The above-described use of thermospikes leads to a significantly better conversion rates in the bisulfite conversion reaction, even when the above-described denaturing solvents are not utilized.

According to a preferred embodiment, the method of the invention is a method, wherein bisulfite treated DNA is subjected directly to methods in the field of methylation analysis. This is especially preferred in view of the avoidance of cross-contaminations in PCR based methods. This embodiment is basically carried out as described in U.S. Ser. No. 11/248,721 (this reference is incorporated by reference to its entirety). According to this, decontaminated DNA is provided which is suitable for DNA methylation analysis. This embodiment is characterized in that DNA is incubated with a bisulfite reagent comprising solution as described above. This leads to a sulfonation, a deamination, or both of unmethylated cytosine. Deamination is a spontaneous process in an aqueous solution and leads to sulfonated uracil comprising DNA. No desulfonation occurs yet.

In a separate step, the DNA comprising sulfonated uracil is brought into contact and incubated with an enzyme which specifically degrades non-sulfonated uracil containing nucleic acids. Such an enzyme is for example Uracil-DNA-Glycosylase (UNG).

In a preferred embodiment for providing a decontaminated template DNA for polymerase based amplification reactions, the sulfonated and/or deaminated template DNA are mixed with an UNG activity and components required for a polymerase mediated amplification reaction or an amplification based detection assay. After degradation of non-sulfonated uracil containing nucleic acids by use of UNG, the UNG activity is terminated and the template DNA is desulfonated by increased temperature. Subsequently the template DNA is ready to be amplified.

In a preferred embodiment, degradation, termination, desulfonation and amplification occur in a single tube during a polymerase based amplification reaction and/or an amplification based assay. Preferably such an amplification is performed in the presence of dUTP instead of dTTP.

In a preferred embodiment, sulfonated and partially or completely deaminated DNA after bisulfite treatment is subjected directly to a polymerase based amplification reaction and/or an amplification based assay without any prior desulfonation. The desulfonation occurs during the initial temperature increase of the amplification reaction.

These particular embodiments have the advantage in comparison to known methods of bisulfite treatment that the purification step after bisulfite treatment becomes dispensable. This is a simplification which results in reduction of costs and handling effort, minimizes loss of bisulfite treated DNA and is also time saving.

In an embodiment, the method of the invention is a method, wherein treating DNA with a reagent or enzyme allowing differentiation of the methylation status comprises purifying the treated DNA.

According to an embodiment, the treatment that leads to a conversion of unmethylated cytosine to uracil while methylated cytosines remain unchanged comprises the purification of the bisulfite treated DNA. According to an embodiment, such a purification comprises a desulfonation of the bisulfite treated DNA by bringing the said into contact with an alkaline reagent or solution for example but not limited to a alkaline solution of about 0.1 mol/l sodium hydroxide.

In a preferred embodiment, the method of the invention is a method, wherein purifying the treated DNA comprises the use of at least one selected from the group comprising: ultrafiltration, Microcon filter device, filter device, ethanol, propanol, silica surface, silica membrane, magnetic particle, polystyrol particle, positively charged surface, and positively charged membrane, charged membrane, charged surface, charged switch membrane, charged switched surface.

In a preferred embodiment, the detection or quantification reaction is carried out by one or more means selected from the group comprising: antibody, western blot analysis, chromatography, immunoassay, ELISA immunoassay, radioimmunoassay, FPLC, HPLC, UV light, light, spectrometer, mass-spectroscopy, MALDI-TOF, nucleic acid, DNA, PNA, oligonucleotide, DNA analogs comprising oligomers, DMA analogs like PNA-monomers, LNA, LNA monomers, Phosphothioates, Methylphophonates, amplification method, PCR method, isothermal amplification method, TMA (transcription mediated amplification), NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, and Ms-SNuPE (Methylation-sensitive Single Nucleotide Primer Extension) method. A person skilled in the art knows to generate and apply such or correspondent means. He also knows how to adjust them according to the invention.

In a particular preferred embodiment the detection or quantification reaction comprises the use of at least one of the following methods or combinations thereof: amplification method, PCR method, isothermal amplification method, NASBA method, LCR method, methylation specific amplification method, MSP (Methylation Specific PCR) method, nested MSP method, HeavyMethyl™ method, detection method, agarose gel, staining of an agarose gel, methylation specific detection method, bisulfite sequencing method, detection by means of DNA-arrays, detection by means of oligonucleotide microarrays, detection by means of CpG-island-microarrays, detection by means of restriction enzymes, simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, and Ms-SNuPE (Methylation-sensitive Single Nucleotide Primer Extension) method.

According to an embodiment, the amplification method can be any kind of amplification method. A person skilled in the art is in knowledge of suitable amplification methods. According to a preferred embodiment, the amplification method is a PCR method. A person skilled in the art knows suitable PCR methods which can be used according to the invention. According to a preferred embodiment, the amplification method is an isothermal amplification. Suitable amplification methods for use according to the invention are well known in the art. Such a method can be for example but not limited to it the Primer Extension method. According to a preferred embodiment, the amplification method is a NASBA method. NASBA methods are RNA-DNA based amplification methods which comprise the use of a Reverse Transcriptase, a RNA polymerase and a RNase. A person skilled in the art is aware of NASBA methods which can be used according to the invention. According to a preferred embodiment, the amplification method is a Ligase Chain Reaction method. In general, these are amplification methods which are based on the use of a ligase. A person skilled in the art knows suitable LCR which can be used according to the invention.

According to an embodiment, the amplification method is a methylation specific amplification. Suitable methylation specific amplification methods are known to those skilled in the art. According to a preferred embodiment, the methylation specific amplification method is the Methylation Specific PCR (MSP) method. The MSP method allows the assessing of the methylation status of virtually any group of CpG sites within a CpG island, independent of the use of methylation-sensitive restriction enzymes (Herman et al. Proc. Natl. Acad. Sci. USA 93:9821-9826,1996; U.S. Pat. No. 5,786,146; these references are incorporated by reference to their entirety). Briefly, DNA is modified by sodium bisulfite converting all unmethylated, but not methylated cytosines to uracil, and subsequently amplified with primers specific for methylated versus unmethylated DNA. MSP primer pairs contain at least one primer, which hybridizes to a bisulfite treated CpG dinucleotide. Therefore, the sequence of said primers comprises at least one CpG dinucleotide. MSP primers specific for non-methylated DNA contain a “T” at the 3′ position of the C position in the CpG. Preferably, therefore, the base sequence of said primers is required to comprise a sequence having a length of at least 9 nucleotides which hybridizes to the bisulfite converted nucleic acid sequence, wherein the base sequence of said oligomers comprises at least one CpG dinucleotide. MSP requires only small quantities of DNA and is sensitive to 0.1% methylated alleles of a given CpG island locus. Bisulfite treatments and amplification method described herein may be used in combination with this detection method.

According to a preferred embodiment, the amplification is a nested MSP method. The nested MSP method is essentially carried out as described in WO 02/18649 and US 20040038245 (these references are incorporated by reference to their entirety). This MSP method considers the apparent conflict of requiring high specificity of the MSP primer to sufficiently differentiate between CG and TG positions and of allowing a mismatch in order to create a unique restriction site. It comprises the expanding of copy numbers of the genetic region of interest. Therefore a polymerase chain reaction is used to amplify a portion of said region wherein the methylation of interest resides. Thereby an amplification product is generated. An aliquot of said product is then used in a second, methylation-specific, polymerase chain reaction to detect the presence of methylation. In other words a non methylation specific PCR is performed prior to the methylation specific PCR.

According to a preferred embodiment, the amplification method is the HeavyMethyl™ method. The HeavyMethyl™ method is essentially carried out as described in WO 02/072880 and Cottrell S E et al. Nucleic Acids Res. Jan. 13, 2004;32(1):e10 (these references are incorporated by reference to their entirety). This method comprises the use of blocking probe oligonucleotides which may be hybridized to the bisulfite treated template nucleic acid concurrently with the PCR primers. Preferably, the blocking oligonucleotides are characterized in that their base sequence comprises a sequence having a length of at least 9 nucleotides which hybridizes to the chemically treated nucleic acid sequence. Thereby the base sequence of said blocker oligonucleotides comprises at least one CpG, TpG or CpA dinucleotide. The amplification of the template nucleic acid is suppressed in case the complementary sequence of the blocking probe is present in the template. In such a case the amplification is terminated at the 5′ position of the blocking probe. The blocking probe may be designed to hybridize to the bisulfite treated nucleic acid in a methylation status specific manner. For example, methylated nucleic acids within a population of unmethylated nucleic acids can be detected by suppressing the amplification of nucleic acids which are unmethylated at a position in question. Therefore a blocking probe would comprise a ‘CpA’ or ‘TpA’ at the position in question, as opposed to a ‘CpG’ if the suppression of amplification of methylated nucleic acids is desired. The use of blocker oligonucleotides requires for a efficient disruption of polymerase-mediated amplification that the blocker oligonucleotides can not be elongated by the polymerase. According to the HeavyMethyl™ method, this is achieved through the use of blockers that are 3′-deoxyoligonucleotides, or oligonucleotides derivatized at the 3′ position with other than a “free” hydroxyl group. For example, but not limited to it, 3′-O-acetyl oligonucleotides are representative of a preferred class of blocker molecules.

Additionally, polymerase-mediated degradation of the blocker oligonucleotides should be precluded. Preferably, such preclusion comprises either i) the use of a polymerase lacking 5′-3′ exonuclease activity, or ii) the use of modified blocker oligonucleotides. These modified blocker oligonucleotides are characterized in having, for example, thioate bridges at the 5′-terminii. This renders the blocker molecule nuclease-resistant. Particular applications may not require such 5′ modifications of the blocker oligonucleotide. For example, degradation of the blocker oligonucleotide will be substantially precluded if the blocker- and primer-binding sites overlap. Thereby the binding of the primer is precluded (e.g., in case of excess blocker oligonucleotide). Therefore the polymerase can not bind on the primer and elongated it. Because no polymerase is extending the primer, the blocking oligonucleotide will not be degraded. A particularly preferred embodiment of the HeavyMethyl™ method, for purposes of the present invention and as implemented herein, comprises the use of peptide nucleic acid (PNA) oligomers as blocking oligonucleotides. Such PNA blocker oligomers are ideally suited because they are neither degraded nor extended by the polymerase.

According to an embodiment, the detection method can be any kind of detection method. A person skilled in the art is in knowledge of suitable detection methods. Preferably, a detection method can be any kind of detection method which comprises the use of a fluorescent dye, a non-fluorescent dye, a mass label, a separation by size, or a separation by weight. For example, but not limited to it, the detection method is a separation by size in an agarose gel followed by a staining of DNA by means of a fluorescent dye. According to a preferred embodiment, the detection method is a methylation specific detection. A person skilled in the art knows suitable methylation specific detection methods. According to a preferred embodiment, the methylation specific detection method is a bisulfite sequencing method. The bisulfite sequencing method is essentially carried out as described in Frommer et al. Proc. Natl. Acad. Sci. USA 89:1827-1831, 1992. The bisulfite sequencing method is a method wherein the sequencing of a previously amplified fragment of the bisulfite treated genomic DNA is carried out. As the bisulfite treated DNA is amplified before sequencing, an amplification method as described herein may be used in combination with this detection method. It is further especially preferred that the results of a bisulfite sequencing are essentially analyzed as described in EP 02090203.7 (this cited reference is incorporated by reference to its entirety). In brief, according to this method the degree of methylation of a cytosine is determined by means of an electropherogram of one or more bases. Thereby the area underneath the electropherogram of a detected base is calculated. The degree of methylation is then deduced by comparison this value for a cytosine position to be analyzed with the value obtained for an unmethylated cytosine. For better results, the determination and the consideration of the conversion rate of cytosine to uracil of the bisulfite treatment and/or a standardization of electropherogram signals is favorable.

According to a preferred embodiment, the detection method is a method of detection by means of a DNA-array. A person skilled in the art knows at lot of suitable DNA-arrays. Preferably, a DNA array comprises DNA molecules which are bound to or elsewise associated with a solid phase. The array can be characterized, for example but not limited to it, in that the DNA molecules are arranged on the solid phase in the form of a rectangular or hexagonal lattice. Thereby the solid phase is at least one phase selected from the group comprising: silicon, glass, polystyrene, aluminum, steel, iron, copper, nickel, silver, gold, nitrocellulose, or plastics such as but not limited to it nylon. But also combinations of the said materials are thinkable. For detection, the DNA hybridized on the array is labeled, preferably with a fluorescent dye. Such labelling is for example, but not limited to it, the simple attachment of Cy3 and Cy5 dyes to the 5′-OH of the DNA fragment. The detection of the fluorescence of the hybridized DNA may be carried out, for example, but not limited to it, via a confocal microscope.

According to a particular preferred embodiment, the detection method is a method of detection by means of a oligonucleotide microarray. An overview of the prior art in oligomer array manufacturing can be gathered from a special edition of Nature Genetics (Nature Genetics Supplement, Volume 21, January 1999, and from the literature cited therein; this reference is incorporated according to its entirety as well as the therein cited references).

According to a particular preferred embodiment, the detection method is a method of detection by means of a CpG-island-microarray. Thereby the immobilized or associated DNA of the array comprises sequences which were derived from CpG islands.

According to a particular preferred embodiment, the detection method is a method of detection by means of a DNA-array as essentially described in WO 99/28498, WO 01/38565, or in WO 02/18632 (these references are incorporated by reference to their entirety).

According to a preferred embodiment, the detection method is a method of detection by means of restriction enzymes. A person skilled in the art is in knowledge of suitable methods.

According to a preferred embodiment, the methylation specific amplification and the detection are carried out simultaneously. Suitable methods are known to those skilled in the art. According to a particular preferred embodiment, the method for simultaneous methylation specific amplification and detection is the COBRA method. The COBRA method is a quantitative methylation method useful for determining DNA methylation levels at specific gene loci in small amounts of genomic DNA (Xiong & Laird, Nucleic Acids Res. 25:2532-2534, 1997; this reference is incorporated by reference to its entirety). According to the COBRA method, restriction enzyme digestion is used to reveal methylation-dependent sequence differences in PCR products of bisulfite-treated DNA. Methylation-dependent sequence differences are first introduced into the genomic DNA by bisulfite treatment. PCR amplification of the bisulfite converted DNA is then performed using methylation unspecific primers followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. Additionally, restriction enzyme digestion of PCR products amplified from bisulfite-converted DNA is also used, in the method described by Sadri & Hornsby (Nucl. Acids Res. 24:5058-5059, 1996; this reference is incorporated by reference to its entirety). Bisulfite treatments and amplification methods described herein may be used in combination with this detection method.

According to a particular preferred embodiment, the method for simultaneous methylation specific amplification and detection is a real-time PCR method. A person skilled in the art knows suitable real-time PCR methods. According to a particular preferred embodiment, the real-time PCR method is a HeavyMethyl™ method. The HeavyMethyl™ method is thereby performed as described above by means of a real-time PCR machine.

According to a particular preferred embodiment, the real-time PCR method is a MethyLight™ method. The MethyLight™ method is a high-throughput quantitative methylation method that utilizes fluorescence-based real-time PCR (TaqMan™) technology that requires no further manipulations after the PCR step (Eads et al., Cancer Res. 59:2302-2306, 1999). Briefly, the MethyLight™ process begins with a mixed sample of genomic DNA that is converted, in a bisulfite reaction, to a mixed pool of methylation-dependent sequence differences according to standard procedures. Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both. The MethyLight™ method may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique also named MSP MethyLight™ method), or with oligonucleotides covering potential methylation sites.

The MethyLight™ process can be used with a “TaqMan®” probe in the amplification process. For example, double-stranded genomic DNA is treated with bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan®) probe. The Taq polymerase 5′ to 3′ endonuclease activity will then displace the TaqMan®) probe by digesting it to release the fluorescent reporter molecule for quantitative detection of its now unquenched signal using a real-time fluorescent detection system.

Variations on the TaqMan® detection technology that are also suitable include the use of dual-probe technology (LightCycler™), fluorescent amplification primers (Sunrise™ technology), Molecular Beacon Probes (Tyagi S., and Kramer F. R., Nature Biotechnology 14, 303-308, 1996), Scorpion primers (Whitcombe et al., Nature and Biotechnology, 17, 804-807, 1999), or LNA (Locked Nucleid Acid) Double-Dye Oligonucleotide probes (Exiqon A/S). All of these techniques may be adapted in a manner suitable for use with bisulfite treated DNA, and moreover in the field of methylation analysis within CpG dinucleotides.

Bisulfite treatments and amplification methods described herein may be used in combination with the MethyLight™ method or its variants.

According to a particular preferred embodiment, the real-time PCR method is the MethyLight™ ALGO™ method. The MethyLight™ ALGO™ method is an improved method of the MethyLight™ method as essentially described in EP 04090255,3 (this reference is incorporated by reference to its entirety). According to this improved method, the degree of methylation is calculated from the signal intensities of probes using different algorithms.

According to a particular preferred embodiment, the real-time PCR method is the QM (quantitative methylation) assay. This assay is a methylation unspecific and therefore unbiased real-time PCR amplification. It is accompanied by the use of two methylation specific probes (MethyLight™) one for the methylated amplificate and a second for the unmethylated amplificate. In this way, two signals are generated which can be used a) to determine the ratio of methylated (CG) to unmethylated (TG) nucleic acids, and at the same time b) to determine the absolute amount of methylated nucleic acids. For the later, a calibration of the assay is necessary with a known amount of control DNA.

According to preferred embodiment, the method for simultaneous methylation specific amplification and detection is a Headloop PCR method. The Headloop PCR method is a suppression PCR method. It essentially carried out as described in Rand K. N., et al., Nucleic Acid Research, 33(14), e127 (this reference is incorporated by reference to its entirety). It is a PCR method for distinguishing related sequences in which the selectivity of amplification is dependent from the amplicon's sequence. A 5′ extension is included in one (or both) primer(s) that corresponds to sequences within one of the related amplicons. After copying and incorporation into the amplificate this sequence is then able to loop back, anneal to the internal sequences and prime to form a hairpin structure. This structure prevents then further amplification. Thus, amplification of sequences containing a perfect match to the 5′ extension is suppressed while amplification of sequences containing mismatches or lacking the sequence is unaffected.

According to a particular preferred embodiment, the method for simultaneous methylation specific amplification and detection is a combination of the Headloop PCR method and the MethyLight™ method, also named Headloop MethyLight™ method.

According to preferred embodiment, the method for simultaneous methylation specific amplification and detection is a Scorpion™ method. This method was first described by Whitcombe et al.: Detection of PCR products using self-probing amplicons and fluorescence. Nat Biotechnol. 1999; 17(8):804-7; Thelwell et al.: Mode of action and application of Scorpion™ primers to mutation detection. Nucleic Acids Res. Oct. 1, 2000;28(19):3752-61; U.S. Pat. No. 6,326,145; U.S. Pat. No. 6,365,729; US 20030087240 A1; these references are incorporated by reference to their entirety). Several embodiments of this method are known to those skilled in the art. All of these methods have the intramolecular probing in common. According to the so-called Hairloop variant, Scorpion™ primers posses a specific probe sequence at their 5′ end. This sequence is present in a hairloop like configuration. A fluorescent dye and a quencher are located in spatial proximity at the end of the probing sequence. After denaturation subsequent to an amplification cycle, the probe hybridizes intramolecularly onto the elongated primer sequence of the same strand. Thereby the hairloop is opened, the dye and the quencher are separated and thus the dye's signal can be detected.

Other Scorpion™ method variants are for example the Duplex variant (Solinas et al.: Duplex Scorpion™ primers in SNP analysis and FRET applications. Nucleic Acids Res. Oct. 15, 2001;29(20):E96), or the variants as described in U.S. Pat. No. 6,326,145 and US 20030087240 (all references are incorporated by reference to their entirety).

According to a particular preferred embodiment, the Scorpion™ method is a method as essentially described in WO 05/024056 (this reference is incorporated by reference to its entirety).

According to a particular preferred embodiment, the method for simultaneous methylation specific amplification and detection is a combination of the HeavyMethyl™ method and the Scorpion™ method, also named HeavyMethyl™ Scorpion™ method.

According to a particular preferred embodiment, the method for simultaneous methylation specific amplification and detection is a combination of the HeavyMethyl™ method and the MethyLight™ method, also named HeavyMethyl™ MethyLight™ method.

According to a particular preferred embodiment, the method for simultaneous methylation specific amplification and detection is a combination of the MSP method and the Scorpion™ method, also named MSP Scorpion™ method.

According to a particular preferred embodiment, the method for simultaneous methylation specific amplification and detection is a combination of the Headloop method and the Scorpion™ method, also named Headloop Scorpion™ method.

According to a preferred embodiment, the method for simultaneous methylation specific amplification and detection is a method of methylation specific primer extension. A person skilled in the art knows several methods which can be used according to the invention.

According to a particular preferred embodiment, the method of methylation specific primer extension is the Ms-SNuPE (methylation-sensitive Single Nucleotide Primer Extension) method. The Ms-SNuPE method is a method as essentially carried out as described in Gonzalgo et al., Nucleic Acids Research 25(12), 2529-2531, 1997 and U.S. Pat. No. 6,251,594 (these references are incorporated by reference to their entirety). According to the Ms-SNuPE method, regions of interest are amplified by PCR from bisulfite treated DNA. After purification of the PCR products, primers are proximately hybridized in front of the position to be analyzed. The primer is then elongated by a single nucleotide either with labeled dCTP or with differently labeled dTTP. In case the cytosine in the original DNA was methylated, then dCTP will be incorporated because methylated cytosines remain unchanged during bisulfite treatment. In the other case, the cytosine in the original DNA was unmethylated, then dTTP will be incorporated because unmethylated cytosine is converted to uracil by bisulfite treatment and subsequent PCR will substitute uracil by thymine. By detection of the different labels, it can be distinguished if a cytosine of a CpG position was methylated or unmethylated. The MS-SNuPE method can also be performed in a quantitative manner.

According to a particular preferred embodiment, the method of methylation specific primer extension is a method as essentially described in WO 01/062960, WO 01/062064, or WO 01/62961 (these references are incorporated by reference to their entirety). All of these methods can be performed in a quantitative manner. According to WO 01/062960, the primer to be extended hybridizes with its 3′ terminus complete or only partially onto the positions of interest. An extension of at least one nucleotide occurs only if the primer hybridizes completely. WO 01/062064 discloses a method in which the primer to be extended hybridizes proximately adjacent or at a distance of up to ten bases to the position to be analyzed. The primer is then extended by at least a single nucleotide. The third method is described in WO 01/62961. According to this method, two sets of oligonucleotides are hybridized to the amplified DNA after bisulfite treatment. The first type of oligonucleotide hybridizes 5′ proximately adjacent or at a distance of up to 10 bases to the position to be analyzed. The second type of oligonucleotide hybridizes on the amplified DNA so that its 5′ terminus hybridizes 3′ proximately adjacent to said position to be analyzed. Through this, the two oligonucleotides are separated from each other by a gap of in the range of 1 to 10 nucleotides. The first type of oligonucleotide is then extended by means of a polymerase, wherein not more than the number of nucleotides lying between the two oligonucleotides are added. Thereby nucleotides are used which comprise differentially labeled dCTP and/or dTTP. The two oligonucleotides are then linked to each other by means of a ligase enzyme. In case the cytosine in the original DNA was methylated, then dCTP will be incorporated. In case the cytosine in the original DNA was unmethylated, then dTTP will be incorporated.

Of course other similar methods, which are further developed methods of the named methods or combinations thereof are also useable according to the invention.

In a preferred embodiment the detection or quantification reaction comprises a nucleic acid; DNA; DNA comprising DNA analog like PNA, LNA, HNA (hexol nucleic acid), phosphothioate, methylphosphonate; oligonucleotide; or PNA oligomer; which
is at least of about 10, about 15, about 20, about 25, about 30, about 35, about 40, about 50, about 60, about 70, about 80, about 90, about 100, about 150 or about 200 nucleotides in length,
has a content of cytosin-nucleotides and guanosin-nucleotides of about 15%, about 20%, about 30%, about 40%, about 50%, about 60%, about 70%, about 80%, or about 85%;
has a melting temperature of about 37° C., about 45° C., about 50° C., about 55° C., about 60° C., about 65° C., about 70° C., about 75° C., about 80° C., about 85° C., about 90° C., about 95° C., or about 99° C.; or
combinations thereof.

In a preferred embodiment the detection or quantification reaction comprises a nucleic acid; DNA; DNA comprising DNA analog like PNA, LNA, HNA (hexol nucleic acid), phosphothioate, methylphosphonate; oligonucleotide; or PNA oligomer; which
is at least of about 10, about 15, about 20, about 25, about 30, about 35, about 40, about 50, about 60, about 70, about 80, about 90, about 100, about 150 or about 200 nucleotides in length,
has a content of cytosin-nucleotides and guanosin-nucleotides of about 15%, about 20%, about 30%, about 40%, about 50%, about 60%, about 70%, about 80%, or about 85%; and
has a melting temperature of about 37° C., about 45° C., about 50° C., about 55° C., about 60° C., about 65° C., about 70° C., about 75° C., about 80° C., about 85° C., about 90° C., about 95° C., or about 99° C.

In a preferred embodiment the detection or quantification reaction comprises a nucleic acid; DNA; DNA comprising DNA analog like PNA, LNA, HNA (hexol nucleic acid), phosphothioate, methylphosphonate; oligonucleotide; or PNA oligomer; which
is free of cytosine;
is free of guanine;
hybridizes onto cytosine-free, guanin-free, or cytosine-free and guanin-free sites of the identifier;
is at least of about 10, about 15, about 20, about 25, about 30, about 35, about 40, about 50, about 60, about 70, about 80, about 90, about 100, about 150 or about 200 nucleotides in length,
has a content of cytosin-nucleotides and guanosin-nucleotides of about 15%, about 20%, about 30%, about 40%, about 50%, about 60%, about 70%, about 80%, or about 85%;
has a melting temperature of about 37° C., about 45° C, about 50° C., about 55° C., about 60° C., about 65° C., about 70° C., about 75° C., about 80° C., about 85° C., about 90° C., about 95° C., or about 99° C.; or
combinations thereof.

In a preferred embodiment the detection or quantification reaction comprises a nucleic acid; DNA; DNA comprising DNA analog like PNA, LNA, HNA (hexol nucleic acid), phosphothioate, methylphosphonate; oligonucleotide; or PNA oligomer; which
is free of cytosine;
is free of guanine;
hybridizes onto cytosine-free, guanin-free, or cytosine-free and guanin-free sites of the identifier;
is at least of about 10, about 15, about 20, about 25, about 30, about 35, about 40, about 50, about 60, about 70, about 80, about 90, about 100, about 150 or about 200 nucleotides in length,
has a content of cytosin-nucleotides and guanosin-nucleotides of about 15%, about 20%, about 30%, about 40%, about 50%, about 60%, about 70%, about 80%, or about 85%; and
has a melting temperature of about 37° C., about 45° C, about 50° C., about 55° C., about 60° C., about 65° C., about 70° C., about 75° C., about 80° C., about 85° C., about 90° C., about 95° C., or about 99° C.

In another preferred embodiment the detection or quantification reaction comprises a nucleic acid; DNA; DNA comprising DNA analog like PNA, LNA, HNA (hexol nucleic acid), phosphothioate, methylphosphonate; oligonucleotide; or PNA oligomer; which
comprises cytosine;
comprises guanine;
hybridizes onto a site covering converted cytosine positions;
is at least of about 10, about 15, about 20, about 25, about 30, about 35, about 40, about 50, about 60, about 70, about 80, about 90, about 100, about 150 or about 200 nucleotides in length;
has a content of cytosin-nucleotides and guanosin-nucleotides of about 15%, about 20%, about 30%, about 40%, about 50%, about 60%, about 70%, about 80%, or about 85%;
has a melting temperature of about 37° C., about 45° C., about 50° C., about 55° C., about 60° C., about 6520 C., about 70° C., about 75° C., about 80° C., about 85° C., about 90° C., about 95° C., or about 99° C., or
combinations thereof.

In another preferred embodiment the detection or quantification reaction comprises a nucleic acid; DNA; DNA comprising DNA analog like PNA, LNA, HNA (hexol nucleic acid), phosphothioate, methylphosphonate; oligonucleotide; or PNA oligomer; which
comprises cytosine;
comprises guanine;
hybridizes onto a site covering converted cytosine positions;
is at least of about 10, about 15, about 20, about 25, about 30, about 35, about 40, about 50, about 60, about 70, about 80, about 90, about 100, about 150 or about 200 nucleotides in length;
has a content of cytosin-nucleotides and guanosin-nucleotides of about 15%, about 20%, about 30%, about 40%, about 50%, about 60%, about 70%, about 80%, or about 85%; and
has a melting temperature of about 37° C., about 45° C., about 50° C., about 55° C., about 60° C., about 65° C., about 70° C., about 75° C., about 80° C., about 85° C., about 90° C., about 95° C., or about 99° C.

In a preferred embodiment, the detection or quantification reaction comprises an oligonucleotide which
is at least of about 5, about 10, about 15, about 20, about 25, about 30, about 35, about 40, about 50, about 60, about 70, about 80, or about 90 nucleotides in length;
has a content of cytosin-nucleotides and guanosin-nucleotides of about 5%, of about 10%, about 20%, about 30%, about 40%, about 50%, about 60%, about 70%, about 80%, about 90% or about 95%;
has a melting temperature of about 37° C., about 45° C., about 50° C., about 55° C., about 60° C., about 65° C., about 70° C., about 75° C., about 80° C., about 85° C., about 90° C., about 95° C., or about 99° C.; or
combinations thereof.

In a preferred embodiment, the detection or quantification reaction comprises an oligonucleotide which
is at least of about 5, about 10, about 15, about 20, about 25, about 30, about 35, about 40, about 50, about 60, about 70, about 80, or about 90 nucleotides in length;
has a content of cytosin-nucleotides and guanosin-nucleotides of about 5%, of about 10%, about 20%, about 30%, about 40%, about 50%, about 60%, about 70%, about 80%, about 90% or about 95%; and
has a melting temperature of about 37° C., about 45° C., about 50° C., about 55° C., about 60° C., about 65° C., about 70° C., about 75° C., about 80° C., about 85° C., about 90° C., about 95° C., or about 99° C.

In a preferred embodiment, the detection or quantification reaction comprises an oligonucleotide which
is cytosine-free or guanine-free or comprises no CpG dinucleotide;
is at least of about 5, about 10, about 15, about 20, about 25, about 30, about 35, about 40, about 50, about 60, about 70, about 80, or about 90 nucleotides in length;
has a content of cytosin-nucleotides and guanosin-nucleotides of about 5%, of about 10%, about 20%, about 30%, about 40%, about 50%, about 60%, about 70%, about 80%, about 90% or about 95%;
has a melting temperature of about 37° C., about 45° C., about 50° C., about 55° C., about 60° C., about 65° C., about 70° C., about 75° C., about 80° C., about 85° C., about 90° C., about 95° C., or about 99° C.; or
combinations thereof.

In a preferred embodiment, the detection or quantification reaction comprises an oligonucleotide which
is cytosine-free or guanine-free or comprises no CpG dinucleotide;
is at least of about 5, about 10, about 15, about 20, about 25, about 30, about 35, about 40, about 50, about 60, about 70, about 80, or about 90 nucleotides in length;
has a content of cytosin-nucleotides and guanosin-nucleotides of about 5%, of about 10%, about 20%, about 30%, about 40%, about 50%, about 60%, about 70%, about 80%, about 90% or about 95%; and
has a melting temperature of about 37° C., about 45° C., about 50° C., about 55° C., about 60° C., about 65° C., about 70° C., about 75° C., about 80° C., about 85° C., about 90° C., about 95° C., or about 99° C.

In another preferred embodiment, the detection or quantification reaction comprises an oligonucleotide which
comprises cytosine, guanine, or both;
hybridizes onto sites covering converted cytosine positions;
is at least of about 5, about 10, about 15, about 20, about 25, about 30, about 35, about 40, about 50, about 60, about 70, about 80, or about 90 nucleotides in length;
has a content of cytosin-nucleotides and guanosin-nucleotides of about 5%, of about 10%, about 20%, about 30%, about 40%, about 50%, about 60%, about 70%, about 80%, about 90% or about 95%;
has a melting temperature of about 37° C., about 45° C., about 50° C., about 55° C., about 60° C., about 65° C., about 70° C., about 75° C., about 80° C., about 85° C., about 90° C., about 95° C., or about 99° C.; or combinations thereof.

In another preferred embodiment, the detection or quantification reaction comprises an oligonucleotide which
comprises cytosine, guanine, or both;
hybridizes onto sites covering converted cytosine positions;
is at least of about 5, about 10, about 15, about 20, about 25, about 30, about 35, about 40, about 50, about 60, about 70, about 80, or about 90 nucleotides in length;
has a content of cytosin-nucleotides and guanosin-nucleotides of about 5%, of about 10%, about 20%, about 30%, about 40%, about 50%, about 60%, about 70%, about 80%, about 90% or about 95%; and
has a melting temperature of about 37° C., about 45° C., about 50° C., about 55° C., about 60° C., about 65° C., about 70° C., about 75° C., about 80° C., about 85° C., about 90° C., about 95° C., or about 99° C.

In a preferred embodiment, the detection or quantification reaction comprises an oligonucleotide which
is at least of about 16, about 20, about 25, about 30, about 35, or about 40 nucleotides in length;
has a content of cytosin-nucleotides and guanosin-nucleotides of about 20%, about 30%, about 40%, about 50%, about 60%, about 70%, or about 80%; and
has a melting temperature of about 50° C., about 53° C., about 56° C., about 59° C., or about 62° C.

In a preferred embodiment, the detection or quantification reaction comprises an oligonucleotide which comprises a gene-specific priming sequence and a sequence which hybridizes on a variant of a sequence polymorphism. According to a preferred embodiment, the gene-specific priming sequence is located in the 5′ terminal region and the sequence hybridizing onto the variant of the sequence polymorphism is located in the 3′ terminal region of the said oligonucleotide. According to a particular preferred embodiment, the sequence hybridizing onto the variant of the sequence polymorphism is located in the 5′ terminal region and gene-specific priming sequence is located in the 3′ terminal region of the said oligonucleotide.

In a preferred embodiment, the detection or quantification reaction comprises an oligonucleotide which comprises two domains,
wherein one domain comprises a target-specific priming sequence of about 10, about 15, about 20, about 25, about 30, about 35, or about 40 nucleotides, has a content of cytosin-nucleotides and guanosin-nucleotides of about 20%, about 30%, about 40%, about 50%, about 60%, about 70%, or about 80%, and has a domain melting temperature of about 50° C., about 52° C., about 54° C., about 56° C., about 58° C., about 60° C., or about 62° C.; and
wherein the other domain comprises a unique sequence of about 5, about 10, about 15, about 20, about 25, about 30, about 35, about 40, about 45, or about 50 nucleotides, and has a content of cytosin-nucleotides and guanosin-nucleotides of about 20%, about 30%, about 40%, about 50%, about 60%, about 70%, or about 80%.

According to the herein specified embodiments for detection or quantification, a said oligonucleotide is a DNA oligonucleotide or comprises at least in parts DNA analogs like but not limited to PNA (peptide nucleic acid), LNA (locked nucleic acid), HNA (hexol nucleic acid), phosphothioate DNA, methylphosphonate DNA.

In a preferred embodiment, methylation analysis comprises at least one selected from the group comprising detection of methylation status, detection of methylation level, detection of methylation pattern, detection of methylation pattern level, amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, and Ms-SNuPE (Methylation-sensitive Single Nucleotide Primer Extension) method.

According to an embodiment, the determining of a methylation status of at least one CpG position, determining of at least one methylation pattern, or both comprises the use of at least one of the following methods or combinations thereof: amplification method, PCR method, isothermal amplification method, NASBA method, LCR method, methylation specific amplification method, MSP (Methylation Specific PCR) method, nested MSP method, HeavyMethyl™ method, detection method, agarose gel, staining of an agarose gel, methylation specific detection method, bisulfite sequencing method, detection by means of DNA-arrays, detection by means of oligonucleotide microarrays, detection by means of CpG-island-microarrays, detection by means of restriction enzymes, simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, and Ms-SNuPE (Methylation-sensitive Single Nucleotide Primer Extension) method. A person skilled in the art knows how to perform such methods. Preferably such methods are performed as described above.

According to a particular preferred embodiment, the method of the invention is a method of detection of sample interchange, crosscontamination, or both in the field of methylation analysis, comprising
providing a sample set of at least one biological sample, wherein at least one sample comprises genomic DNA differentially methylated at least at one position,
applying at least one identifier for each sample,
subjecting each sample with at least one identifier to a detection or quantification reaction that is specific for the at least one identifier, and
deducing the presence or absence of a sample interchange, of a crosscontamination, or both from the presence or absence of at least one identifier in a single sample.

In a particular preferred embodiment, the step of deducing the presence or absence of a sample interchange, of a crosscontamination, or both further comprises
deducing the extent of a crosscontamination for a single sample from the absolute or relative amount of at least one identifier present in said single sample.

A particular preferred embodiment comprises further contacting the DNA of each sample and the applied at least one identifier with a reagent or enzyme which differentiates between a methylated or an unmethylated position. Said particular preferred embodiments of detection of sample interchange, crosscontamination, or both in the field of methylation analysis are carried out essentially as the herein described embodiments of identifying at least one biological sample in the field of methylation analysis.

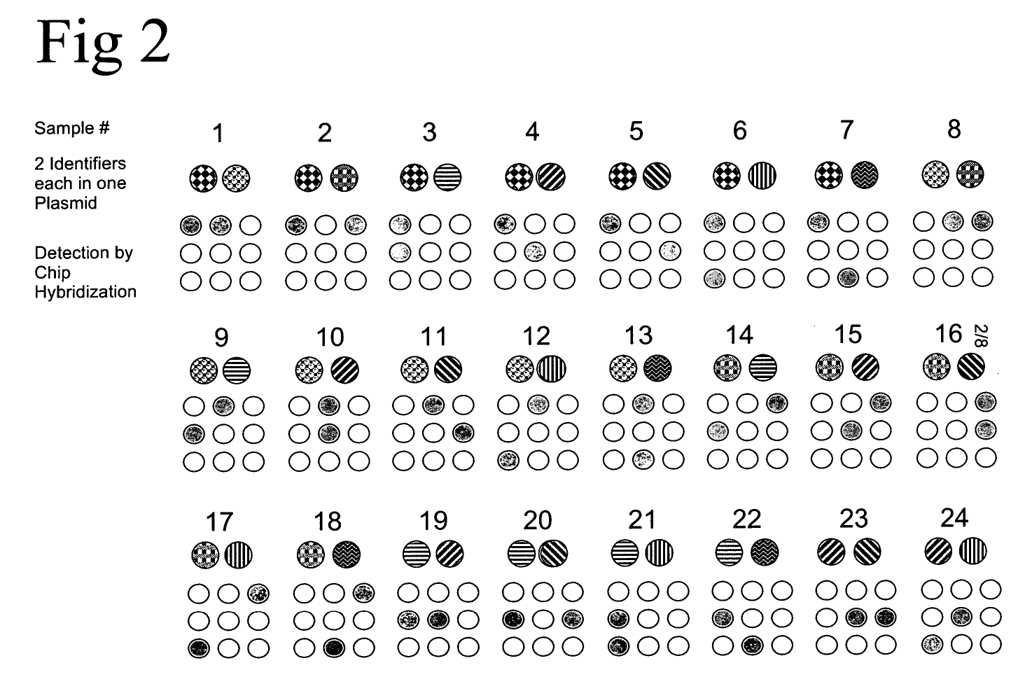

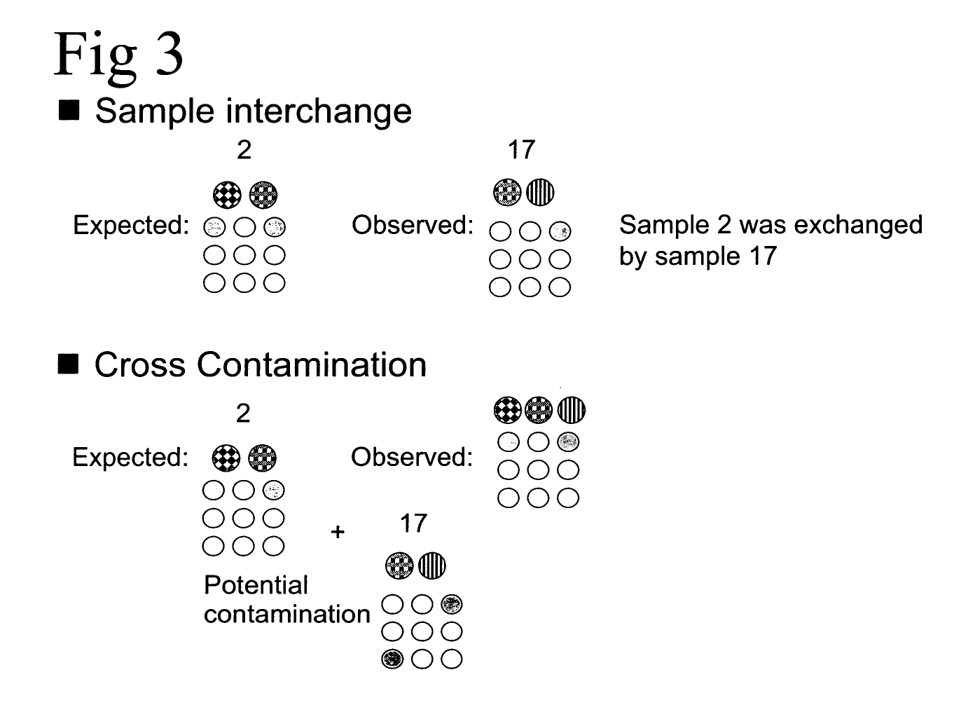

According to a particular preferred embodiment, identifiers are applied to samples of a sample set as indicated by FIG. 2. Each sample of the set is represented by two different identifiers, for example, but not limited to, by two different identifiers belonging to a set of variants of sequence polymorphism. Preferably, thereby each identifier is encoded by a different plasmid. A respective sample can be identified in between steps of an experimental procedure or subsequent to it by amplifying the variants of the sequence polymorphism and hybridizing the amplicon onto a chip. Each combination of identifiers results in a unique hybridization pattern. A sample interchange is detected if two hybridization signals are detected whereby one or both are not characteristic for the applied identifiers for said sample. A sample cross contamination is detected if at least three instead of two hybridization signals are detected. Thereby the non-expected hybridizations signals for said sample indicate with which one or more samples the said sample is contaminated. By quantifying the hybridization signals it is possible to deduce the amount of contamination(s). According to this embodiment, the use of two different identifiers for each sample has the advantage that the assignment of the identifiers to the different samples is unambiguous. For example, in the case that two identical identifiers are used and two different hybridization signals are detected, it can not be distinguished between a cross contamination and a sample to which two different identifiers are applied.

According to a particular preferred embodiment a maximum of 28 samples are encoded by means of the above described system of two different identifiers. According to other particular preferred embodiments, the use of various different numbers of identifiers are used for coding samples. These embodiments are summarized in Table 1.

TABLE 1

| | number of plasmids per sample | | | | | | | |
|---|---|---|---|---|---|---|---|---|
| number of identifier per plasmid | 1 | 2 | 3 | 4 | 5 | 6 | 7 | 8 |
| 1 | 1 | — | — | — | — | — | — | — |
| 2 | 2 | 1 | — | — | — | — | — | — |
| 3 | 3 | 3 | 1 | — | — | — | — | — |
| 4 | 4 | 6 | 4 | 1 | — | — | — | — |
| 5 | 5 | 10 | 10 | 5 | 1 | — | — | — |
| 6 | 6 | 15 | 20 | 15 | 6 | 1 | — | — |
| 7 | 7 | 21 | 35 | 35 | 21 | 7 | 1 | — |
| 8 | 8 | 28 | 56 | 70 | 56 | 28 | 8 | 1 |
| 9 | 9 | 36 | 84 | 126 | 126 | 84 | 36 | 9 |
| 10 | 10 | 45 | 120 | 210 | 252 | 210 | 120 | 45 |
| 11 | 11 | 55 | 165 | 330 | 462 | 462 | 330 | 165 |
| 12 | 12 | 66 | 220 | 495 | 792 | 924 | 792 | 495 |
| 13 | 13 | 78 | 286 | 715 | 1287 | 1716 | 1716 | 1287 |
| 14 | 14 | 91 | 364 | 1001 | 2002 | 3003 | 3432 | 3003 |
| 15 | 15 | 105 | 455 | 1365 | 3003 | 5005 | 6435 | 6435 |
| 16 | 16 | 120 | 560 | 1820 | 4368 | 8008 | 11440 | 12870 |
| 17 | 17 | 136 | 680 | 2380 | 6188 | 12376 | 19448 | 24310 |
| 18 | 18 | 153 | 816 | 3060 | 8568 | 18564 | 31824 | 43758 |
| 19 | 19 | 171 | 969 | 3876 | 11628 | 27132 | 50388 | 75582 |
| 20 | 20 | 190 | 1140 | 4845 | 15504 | 38760 | 77520 | 125970 |
| 21 | 21 | 210 | 1330 | 5985 | 20349 | 54264 | 116280 | 203490 |
| 22 | 22 | 231 | 1540 | 7315 | 26334 | 74613 | 170544 | 319770 |
| 23 | 23 | 253 | 1771 | 8855 | 33649 | 100947 | 245157 | 490314 |
| 24 | 24 | 276 | 2024 | 10626 | 42504 | 134596 | 346104 | 735471 |
| 25 | 25 | 300 | 2300 | 12650 | 53130 | 177100 | 480700 | 1081575 |
| 26 | 26 | 325 | 2600 | 14950 | 65780 | 230230 | 657800 | 1562275 |
| 27 | 27 | 351 | 2925 | 17550 | 80730 | 296010 | 888030 | 2220075 |
| 28 | 28 | 378 | 3276 | 20475 | 98280 | 376740 | 1184040 | 3108105 |
| 29 | 29 | 406 | 3654 | 23751 | 118755 | 475020 | 1560780 | 4292145 |
| 30 | 30 | 435 | 4060 | 27405 | 142506 | 593775 | 2035800 | 5852925 |
| 31 | 31 | 465 | 4495 | 31465 | 169911 | 736281 | 2629575 | 7888725 |
| 32 | 32 | 496 | 4960 | 35960 | 201376 | 906192 | 3365856 | 10518300 |
| 33 | 33 | 528 | 5456 | 40920 | 237336 | 1107568 | 4272048 | 13884156 |
| 34 | 34 | 561 | 5984 | 46376 | 278256 | 1344904 | 5379616 | 18156204 |
| 35 | 35 | 595 | 6545 | 52360 | 324632 | 1623160 | 6724520 | 23535820 |
| 36 | 36 | 630 | 7140 | 58905 | 376992 | 1947792 | 8347680 | 30260340 |
| 37 | 37 | 666 | 7770 | 66045 | 435897 | 2324784 | 10295472 | 38608020 |
| 38 | 38 | 703 | 8436 | 73815 | 501942 | 2760681 | 12620256 | 48903492 |
| 39 | 39 | 741 | 9139 | 82251 | 575757 | 3262623 | 15380937 | 61523748 |
| 40 | 40 | 780 | 9880 | 91390 | 658008 | 3838380 | 18643560 | 76904685 |
| 41 | 41 | 820 | 10660 | 101270 | 749398 | 4496388 | 22481940 | 95548245 |
| 42 | 42 | 861 | 11480 | 111930 | 850668 | 5245786 | 26978328 | 118030185 |
| 43 | 43 | 903 | 12341 | 123410 | 962598 | 6096454 | 32224114 | 145008513 |
| 44 | 44 | 946 | 13244 | 135751 | 1086008 | 7059052 | 38320568 | 177232627 |
| 45 | 45 | 990 | 14190 | 148995 | 1221759 | 8145060 | 45379620 | 215553195 |
| 46 | 46 | 1035 | 15180 | 163185 | 1370754 | 9366819 | 53524680 | 260932815 |
| 47 | 47 | 1081 | 16215 | 178365 | 1533939 | 10737573 | 62891499 | 314457495 |
| 48 | 48 | 1128 | 17296 | 194580 | 1712304 | 12271512 | 73629072 | 377348994 |
| 49 | 49 | 1176 | 18424 | 211876 | 1906884 | 13983816 | 85900584 | 450978066 |
| 50 | 50 | 1225 | 19600 | 230300 | 2118760 | 15890700 | 99884400 | 536878650 |
| 51 | 51 | 1275 | 20825 | 249900 | 2349060 | 18009460 | 115775100 | 636763050 |
| 52 | 52 | 1326 | 22100 | 270725 | 2598960 | 20358520 | 133784560 | 752538150 |
| 53 | 53 | 1378 | 23426 | 292825 | 2869685 | 22957480 | 154143080 | 886322710 |
| 54 | 54 | 1431 | 24804 | 316251 | 3162510 | 25827165 | 177100560 | 1040465790 |
| 55 | 55 | 1485 | 26235 | 341055 | 3478761 | 28989675 | 202927725 | 1217566350 |
| 56 | 56 | 1540 | 27720 | 367290 | 3819816 | 32468436 | 231917400 | 1420494075 |
| 57 | 57 | 1596 | 29260 | 395010 | 4187106 | 36288252 | 264385836 | 1652411475 |
| 58 | 58 | 1653 | 30856 | 424270 | 4582116 | 40475358 | 300674088 | 1916797311 |
| 59 | 59 | 1711 | 32509 | 455126 | 5006386 | 45057474 | 341149446 | 2217471399 |
| 60 | 60 | 1770 | 34220 | 487635 | 5461512 | 50063860 | 386206920 | 2558620845 |
| 61 | 61 | 1830 | 35990 | 521855 | 5949147 | 55525372 | 436270780 | 2944827765 |
| 62 | 62 | 1891 | 37820 | 557845 | 6471002 | 61474519 | 491796152 | 3381098545 |
| 63 | 63 | 1953 | 39711 | 595665 | 7028847 | 67945521 | 553270671 | 3872894697 |
| 64 | 64 | 2016 | 41664 | 635376 | 7624512 | 74974368 | 621216192 | 4426165368 |
| 65 | 65 | 2080 | 43680 | 677040 | 8259888 | 82598880 | 696190560 | 5047381560 |
| 66 | 66 | 2145 | 45760 | 720720 | 8936928 | 90858768 | 778789440 | 5743572120 |
| 67 | 67 | 2211 | 47905 | 766480 | 9657648 | 99795696 | 869648208 | 6522361560 |
| 68 | 68 | 2278 | 50116 | 814385 | 10424128 | 109453344 | 969443904 | 7392009768 |
| 69 | 69 | 2346 | 52394 | 864501 | 11238513 | 119877472 | 1078897248 | 8361453672 |
| 70 | 70 | 2415 | 54740 | 916895 | 12103014 | 131115985 | 1198774720 | 9440350920 |
| 71 | 71 | 2485 | 57155 | 971635 | 13019909 | 143218999 | 1329890705 | 1,0639E+10 |
| 72 | 72 | 2556 | 59640 | 1028790 | 13991544 | 156238908 | 1473109704 | 1,1969E+10 |
| 73 | 73 | 2628 | 62196 | 1088430 | 15020334 | 170230452 | 1629348612 | 1,3442E+10 |
| 74 | 74 | 2701 | 64824 | 1150626 | 16108764 | 185250786 | 1799579064 | 1,5071E+10 |
| 75 | 75 | 2775 | 67525 | 1215450 | 17259390 | 201359550 | 1984829850 | 1,6871E+10 |
| 76 | 76 | 2850 | 70300 | 1282975 | 18474840 | 218618940 | 2186189400 | 1,8856E+10 |
| 77 | 77 | 2926 | 73150 | 1353275 | 19757815 | 237093780 | 2404808340 | 2,1042E+10 |
| 78 | 78 | 3003 | 76076 | 1426425 | 21111090 | 256851595 | 2641902120 | 2,3447E+10 |
| 79 | 79 | 3081 | 79079 | 1502501 | 22537515 | 277962685 | 2898753715 | 2,6089E+10 |
| 80 | 80 | 3160 | 82160 | 1581580 | 24040016 | 300500200 | 3176716400 | 2,8988E+10 |
| 81 | 81 | 3240 | 85320 | 1663740 | 25621596 | 324540216 | 3477216600 | 3,2164E+10 |
| 82 | 82 | 3321 | 88560 | 1749060 | 27285336 | 350161812 | 3801756816 | 3,5641E+10 |
| 83 | 83 | 3403 | 91881 | 1837620 | 29034396 | 377447148 | 4151918628 | 3,9443E+10 |
| 84 | 84 | 3486 | 95284 | 1929501 | 30872016 | 406481544 | 4529365776 | 4,3595E+10 |
| 85 | 85 | 3570 | 98770 | 2024785 | 32801517 | 437353560 | 4935847320 | 4,8125E+10 |
| 86 | 86 | 3655 | 102340 | 2123555 | 34826302 | 470155077 | 5373200880 | 5,306E+10 |
| 87 | 87 | 3741 | 105995 | 2225895 | 36949857 | 504981379 | 5843355957 | 5,8434E+10 |
| 88 | 88 | 3828 | 109736 | 2331890 | 39175752 | 541931236 | 6348337336 | 6,4277E+10 |
| 89 | 89 | 3916 | 113564 | 2441626 | 41507642 | 581106988 | 6890268572 | 7,0625E+10 |
| 90 | 90 | 4005 | 117480 | 2555190 | 43949268 | 622614630 | 7471375560 | 7,7516E+10 |
| 91 | 91 | 4095 | 121485 | 2672670 | 46504458 | 666563898 | 8093990190 | 8,4987E+10 |
| 92 | 92 | 4186 | 125580 | 2794155 | 49177128 | 713068356 | 8760554088 | 9,3081E+10 |
| 93 | 93 | 4278 | 129766 | 2919735 | 51971283 | 762245484 | 9473622444 | 1,0184E+11 |
| 94 | 94 | 4371 | 134044 | 3049501 | 54891018 | 814216767 | 1,0236E+10 | 1,1132E+11 |
| 95 | 95 | 4465 | 138415 | 3183545 | 57940519 | 869107785 | 1,105E+10 | 1,2155E+11 |
| 96 | 96 | 4560 | 142880 | 3321960 | 61124064 | 927048304 | 1,1919E+10 | 1,326E+11 |

Table 1 gives an overview over various particular preferred embodiments. It shows the maximum amount of samples which can be encoded by means of 1, 2, 3, 4, 5, 6, 7, or 8 plasmids and by means of 1 to 96 identifiers, whereby each identifier can be comprised by each plasmid. In general, the following formula expresses the maximum amount of samples which can be encoded by means of a certain number of plasmids and a certain number of identifiers:

$$K ( n , k ) = ( \begin{matrix}n \\ k\end{matrix} )$$

Thereby K represents the maximum number of samples which can be encoded, k represents the number of identifiers (e.g. polymorphism variants), and n represents the number of plasmids. Of course further correspondent embodiments are possible and herewith preferred.

According to a particular preferred embodiment, the method of the invention is a method of identifying a sample in a pooled sample set in the field of methylation analysis, comprising
providing a pooled sample set of at least one biological sample, wherein at least one sample comprises genomic DNA differentially methylated at least at one position;
applying at least one identifier for each sample;
subjecting the sample set to a detection or quantification reaction that is specific for the at least one identifier of each sample; and
identifying a sample in the pooled sample set by detecting the respective applied at least one identifier.

A particular preferred embodiment comprises further contacting the DNA of each sample and the applied at least one identifier with a reagent or enzyme which differentiates between a methylated or an unmethylated position. Said particular preferred embodiments of identifying a sample in a pooled sample set in the field of methylation analysis are carried out essentially as the herein described embodiments of identifying at least one biological sample in the field of methylation analysis.

According to a particular preferred embodiment, samples taken form the same individual are encoded by identifiers of the same set of identifiers. This allows a organ specific encoding of the samples, a tissue specific encoding of the samples, a body specific spacial encoding of the samples, or combinations thereof. According to a particular embodiment, samples taken from different individuals are encoded by identifiers of different sets of identifiers. It is further particular preferred, to combine these two embodiments, so that it is possible to pool the samples derived from the same individual and to analyze them simultaneously with pooled samples of the other individuals. The samples can be assigned to the corresponding individuals by means of the individual specific identifiers. It is possible to identify each sample and to assign it to analysis results by means of the identifiers specific for each sample obtained from the same individual. Therefore it is necessary that each identifier specific for each sample is associated with the genomic DNA or has similar properties as the genomic DNA, in case exogenous identifiers are used. But of course also sample DNA endogeneous identifiers are used and also preferred. Such exogenous or endogeneous identifiers can, for example but not limited to, be all kinds of polymorphisms (sequence, length, deletion, SNP).

According to a particular preferred embodiment, the method of the invention is a method of detection of an amplification inhibition in the field of methylation analysis, comprising
providing a sample set of at least one biological sample, wherein at least one sample comprises genomic DNA differentially methylated at least at one position;
applying at least one identifier for each sample;
subjecting each sample with at least one identifier to an amplification reaction that is specific for the at least one identifier; and
deducing a presence, absence or partial amplification inhibition from the presence, absence, or amount of the product of the identifier specific amplification reaction.

A particular preferred embodiment comprises further contacting the DNA of each sample and the applied at least one identifier with a reagent or enzyme which differentiates between a methylated or an unmethylated position. Said particular preferred embodiments of detection of an amplification inhibition in the field of methylation analysis are carried out essentially as the herein described embodiments of identifying at least one biological sample in the field of methylation analysis.

According to this embodiment, a person skilled in the art can easily detect an amplification inhibition because at least one identifier is applied which can be detected by amplification. He deduces a presence of an amplification inhibition in case the applied identifier(s) is not amplifiable any more.

According to a particular preferred embodiment, the method of the invention is a method of normalization, calibration, or both in the field of methylation analysis, comprising
providing a sample set of at least one biological sample, wherein at least one sample comprises genomic DNA differentially methylated at least at one position;
applying at least one identifier for each sample by adding at least one identifier to each provided sample;
subjecting each sample with at least one identifier to a detection or quantification reaction; and
normalizing at least one sample, calibrating an experimental procedure, or both according to the detected or quantified one or more identifiers compared to the added total amount of the one or more identifiers. Thereby normalization means a correction according to standards, in particular according to the used one or more identifiers. Furthermore, calibration means an adjustment of an experimental procedure or method, so that procedure or method characteristic values lie within certain pre-defined ranges. According to the invention, such characteristic values can be those derived from one or more identifiers.

According to a preferred embodiment, the one or more identifier applied for at least one biological sample are detected or quantified at least twice during an experimental procedure. Preferably, they are detected or quantified after one or more steps of the experimental procedure and subsequent to the experimental procedure.

A particular preferred embodiment comprises further contacting the DNA of each sample and the applied at least one identifier with a reagent or enzyme which differentiates between a methylated or an unmethylated position. The particular preferred embodiments of normalization, calibration, or both in the field of methylation analysis are carried out essentially as the herein described embodiments of identifying at least one biological sample in the field of methylation analysis.

According to this embodiment, the one or more identifiers are applied in a defined amount. It is possible to calibrate an experimental procedure or to normalize samples processed by an experimental procedure by using these identifiers as an internal standard and taking the quantitative changes into consideration. Such changes might be determined, for example but not limited to, by pipetting steps or amplifications.

According to a particular preferred embodiment, the method of the invention is a method of identification of a carry over contamination in the field of methylation analysis, comprising
providing a sample set of at least one biological sample, wherein at least one sample comprises genomic DNA differentially methylated at least at one position;
applying at least one identifier for each sample;
subjecting each sample with at least one identifier to a detection or quantification reaction that is specific for at least one identifier; and
deducing the presence of a sample carry over contamination from the presence of at least one identifier not applied for said sample, or deducing the absence of a sample contamination from the absence of identifiers not applied for said sample.

A particular preferred embodiment comprises further contacting the DNA of each sample and the applied at least one identifier with a reagent or enzyme which differentiates between a methylated or an unmethylated position. Said particular preferred embodiments of identification of a carry over contamination in the field of methylation analysis are carried out essentially as the herein described embodiments of identifying at least one biological sample in the field of methylation analysis.

According to this embodiment, each set of samples is provided with a identifier when it is processed or stored at a certain location. A carry over contamination is existent, in case a previous for another sample set used identifier is detected for a sample set.

According to a particular preferred embodiment, the method of the invention is a method of assessing the success of a hybridization step in the field of methylation analysis, comprising
providing a sample set of at least one biological sample, wherein at least one sample comprises genomic DNA differentially methylated at least at one position;
applying at least one identifier;
subjecting each sample including the applied at least one identifier to a detection or quantification reaction that is specific for the said at least one identifier; wherein the detection or quantification reaction comprises a hybrization step,
assessing the success of the hybridization step wherein (a) the presence of a signal derived for the applied at least one identifier indicates the presence of a successful hybrization step, and wherein (b) the absence of signal derived for the applied at least one identifier indicates the presence of an unsuccessful hybrization step.

A particular preferred embodiment comprises further contacting the DNA of each sample and the applied at least one identifier with a reagent or enzyme which differentiates between a methylated or an unmethylated position. Said particular preferred embodiments of assessing the success of a hybridization step in the field of methylation analysis are carried out essentially as the herein described embodiments of identifying at least one biological sample in the field of methylation analysis.

According to this embodiment, it is in principle sufficient to apply only one identifier per experimental batch. Preferably, two or more identifiers are applied for a single experimental batch. This is in particular the case wherein the processed samples are treated differently or independently from one another. Most preferably, at least one identifier is applied for every sample to be processed per experimental batch.

According to a particular preferred embodiment, the method of the invention is a method of determining the rate of DNA conversion in the field of methylation analysis, comprising
providing a sample set of at least one biological sample, wherein at least one sample comprises genomic DNA differentially methylated at least at one position;
applying at least one identifier for each sample, at least one of the applied identifiers comprises a cytosine that is not part of a CpG dinucleotide;
subjecting each sample with at least one identifier to at least one reaction that converts unmethylated cytosines to a base with a different base pairing behaviour than cytosine, in particular to uracil, while methylated cytosines remain unchanged;
subjecting the at least one identifier of each sample to at least one quantification reaction, wherein the total amount of identifier and the amount of converted identifier are detected; and
determining the rate of DNA conversion according to the amount of converted identifier compared to the total amount of identifier.

According to this embodiment, the conversion rate of a bisulfite treatment is determined by quantifying the amount of an applied identifier before a step and thereafter. Thereby the step comprises contacting the DNA and at least one identifier with a bisulfite reagent. Usually the amount after treatment is divided by the amount before treatment. But further possibilities are possible and also included herewith.

According to other embodiments, the efficiency of an experimental procedure or only steps of it are determined correspondingly as the conversion rate of bisulfite treatment.

According to a particular preferred embodiment, the method of the invention is a method for testing an experimental procedure, comprising
applying at least one identifier instead of a biological sample to an experimental procedure;
subjecting the one or more identifiers to a detection or quantification reaction that is specific for the said one or more identifiers, and that is carried out before or after individual steps of the experimental procedure or subsequent to it.

According to a particular preferred embodiment, the method of the invention is a method for testing an experimental procedure in the field of methylation analysis, comprising
applying at least one identifier instead of a biological sample to an experimental procedure, the experimental procedure comprising contacting the applied at least one identifier with a reagent or enzyme which differentiates between a methylated or an unmethylated position;
subjecting the one or more identifiers to a detection or quantification reaction that is specific for the said one or more identifiers, and that is carried out before or after individual steps of the experimental procedure or subsequent to it.

Accordingly, one or more identifiers substituting a biological sample are referred herein as identifier sample.

In a particular preferred embodiment, one or more identifiers substitute a biological sample. Preferably, one or more identifiers substitute a biological sample within a sample set or one or more identifiers substitute a sample set resulting in an identifier sample set. According to these embodiments, the one or more identifiers are provided in comparable, similar or identical manners as a biological sample. For example, but not limited to, the one or more identifiers are provided in the same container, in the same buffer or both as a biological sample. But, according to these embodiments, the one or more identifiers can also be provided as dried substance or in solution with water or any suitable buffer. According to these embodiments the identifiers are detected or quantified in between individual steps of the experimental procedure or at its end. The detection or quantification reaction is carried out as specified by other embodiments described herein. A person skilled in the art knows to adjust them if necessary. According to this embodiment, the identifier samples or identifier samples sets are subjected to other embodiments described herein.

According to the embodiments described herein, the experimental procedure is for example but not limited to it any combination of chemical, biological or physical reactions.

According to an particular preferred embodiment, the testing of an experimental procedure comprises at least one of the following
determining the probability for a sample interchange, crosscontamination, or both;
determining the extent of a possible crosscontamination;
determining the probability of identifying a sample in a pooled sample set;
determining the probability of an amplification inhibition;
calibrating the experimental procedure;
determining the necessity of normalization;
determining the probability of carry over contamination;
determining the efficiency of a reaction, of a step of said experimental procedure, or of the complete experimental procedure;
optimizing the experimental procedure; and
determining the presence of a successful hybridization step or the presence of an unsuccessful hybridization step.

In a particular preferred embodiment, the testing of an experimental procedure comprises determining the probability for a sample interchange, crosscontamination, or both. According to this embodiment, at least one or more identifiers substitute a biological sample (identifier sample) and at least two identifier samples are applied to an experimental procedure. A sample interchange or crosscontamination is determined by detecting the presence or absence of applied identifiers. A interchange is determined wherein a) at least one of the applied identifiers of an identifier sample is not detected, and wherein b) at least one identifier is detected that was applied to a different identifier sample, said identifiers samples being processed at the same time. A crosscontamination of an identifier sample is determined wherein a) at least one identifier is detected that was applied to a different identifier sample and both identifier samples were processed at the same time, and wherein b) all of the applied identifiers of said identifier sample are detected. A simultaneous interchange and crosscontamination is detected wherein a interchange and a cross contamination are determined for a single identifier sample for the same experimental procedure run. The probability for a sample interchange is determined by multiplying the quotient of the halved number of interchanged identifier samples and the total number of analyzed identifier samples with the factor 100. The probability for a crosscontamination is analogically determined by multiplying the quotient of the number of crosscontaminated identifier samples and the total number of analyzed identifier samples by the factor 100. The probability for simultaneous sample interchange and crosscontamination is also determined analogically by multiplying the quotient of the number of interchanged and crosscontaminated identifier samples and the total number of analyzed identifiers samples by the factor 100. Of course and also preferred, a person skilled in the art is aware of other suitable algorithms.

In an particular preferred embodiment, the testing of an experimental procedure comprises determining the extent of a crosscontamination. According to this embodiment, at least one or more identifiers substitute a biological sample (identifier sample) and at least two identifier samples are applied to an experimental procedure. The extent of a crosscontamination is determined by determining the ratio of the amount of contaminating identifier and the total amount of identifier detected in the quantification reaction for an identifier sample. According to a particular preferred embodiment, a probability distribution for the extent of crosscontaminations of an experimental procedure is determined by considering many crosscontaminated identifier samples. Of course, and also preferred, a person skilled in the art is aware of other suitable algorithms.

In a particular preferred embodiment, the testing of an experimental procedure in the field of methylation analysis comprises determining the probability for a sample interchange, crosscontamination, or both as specified above. According to a particular preferred embodiment, the determining of the probability for a sample interchange, crosscontamination, or both comprises contacting of at least one identifier with a bisulfite reagent.

In a particular preferred embodiment, the testing of an experimental procedure comprises determining the probability of identifying a sample, in particular in a pooled sample set. According to this embodiment, the one or more identifiers are applied instead of a biological sample (identifier sample). Preferably, an arbitrarily number of identifier samples is combined to a pooled sample set. The identifier sample or the preferred identifier sample set is then applied to an experimental procedure. The probability of identifying a sample before or after an individual step of the experimental procedure or at the end of it is determined by performing many times the detection or quantification reaction specific for an identifier, the identifier being part of an identifier sample. The probability is then determined by multiplying the ratio of successful attempts in detecting said identifier and the total attempt in detecting said identifier by the factor 100. Of course, also preferred are other suitable algorithms a person skilled in the art is aware of. These particular embodiments are particular of use for determining the minimum amount of identifier or biological sample which has to be applied to an experimental procedure to obtain reliable results. Preferably, these embodiments are of particular use for determining the amount of identifier or biological sample which allow reliable results with a likelihood of about 60%, about 75%, of about 85%, of about 95%, of about 98%, of about 99%, or about 100%.

In a particular preferred embodiment, the testing of an experimental procedure in the field of methylation analysis comprises determining the probability of identifying a sample, in particular in a pooled sample set as specified above. According to a particular preferred embodiment, the determining of the probability of identifying a sample, in particular in a pooled sample set comprises contacting of at least one identifier with a bisulfite reagent.

In a particular preferred embodiment, the testing of an experimental procedure comprises determining the probability of an amplification inhibition. According to this embodiment, the one or more identifiers are applied to an experimental procedure instead of a biological sample (identifier sample). An amplification inhibition for an identifier sample is present wherein at least one applied identifier is not detected during a previously established amplification based detection or quantification. The probability of an amplification inhibition is determined by multiplying the ratio of the number of unsuccessful detection attempts and the total number of detection attempts by the factor 100. Of course and also preferred, a person skilled in the art is aware of other suitable algorithms. Preferably, for determining the probability of amplification inhibition, one or more identifier samples are applied to the experimental procedure and at least two identifiers are subjected to correspondent previously established amplification based detections or quantifications. The probability of an amplification inhibition is then determined by averaging the probabilities of an amplification inhibition determined for each considered identifier. Of course, also preferred are other suitable algorithms a person skilled in the art is aware of.

In a particular preferred embodiment, the testing of an experimental procedure in the field of methylation analysis comprises determining the probability of an amplification inhibition as specified above. According to a particular preferred embodiment, the determining of the probability of an amplification inhibition comprises contacting of at least one identifier with a bisulfite reagent.

In a particular preferred embodiment, the testing of an experimental procedure comprises calibrating the experimental procedure. According to this embodiment, at least one or more identifiers substitute a biological sample (identifier sample) and at least one identifier sample is applied to an experimental procedure. The calibration of the experimental procedure is realized in amending the different single steps of the experimental procedure so that the applied one or more identifiers are detected with a certain predefined likelihood, that the amount of the applied one or more identifiers determined in the quantification reaction lies within a certain predefined range, or both. The predefined likelihood and the predefined quantification range are determined by at least one run of the optimized experimental procedure. This embodiment is particularly preferred whenever an experimental procedure is established or re-established, for example, but not limited to, after a location change or after a time period not performing the procedure.

In a particular preferred embodiment, the testing of an experimental procedure in the field of methylation analysis comprises calibrating the experimental procedure as specified above. According to a particular preferred embodiment, the calibrating of an experimental procedure comprises contacting of at least one identifier with a bisulfite reagent.

In a particular preferred embodiment, the testing of an experimental procedure comprises determining the necessity of normalization for said experimental procedure. According to this embodiment, at least one or more identifiers substitute a biological sample (identifier sample) and at least one identifier sample is applied to an experimental procedure. The applied identifiers are quantified before or after individual steps of the experimental procedure or at its end. The necessity of normalization is determined wherein the amounts of one or more quantified identifiers is not within a certain predefined range. In many cases, but not limited to them, this range is determined by subsequent data processing, analysis or interpretations and not by the experimental procedure itself. Wherein the necessity is determined, also suitable algorithms for normalization are preferred a person skilled in the art is aware of or is able to adjust.

In a particular preferred embodiment, the testing of an experimental procedure in the field of methylation analysis comprises determining the necessity of normalization for said experimental procedure as specified above. For example but not limited to it, the quantification of the effect of a methylation sensitive digestion can be normalized by one or more applied identifiers containing a recognition site for the used enzyme or enzymes. According to a particular preferred embodiment, the determining the necessity of normalization for an experimental procedure comprises contacting of at least one identifier with a bisulfite reagent.

In a particular preferred embodiment, the testing of an experimental procedure comprises determining the probability of carry over contamination. According to this embodiment, at least one or more identifiers substitute a biological sample (identifier sample), and at least one identifier sample is applied sequentially to at least two experimental procedure runs. Thereby said experimental procedure runs can comprise the same or similar reaction(s) or not. A carry over contamination for an identifier sample is determined wherein an identifier is detected that was applied to a previous run. A sample is considered as free from a carry over contamination wherein no identifier is detected that is indicative for a previous run. The probability of carry over contamination for an experimental procedure is determined by multiplying the ratio of the number of carry over contaminated sample and the total number of samples by the factor 100. Of course and also preferred, a person skilled in the art is aware of other suitable algorithms.

In a particular preferred embodiment, the testing of an experimental procedure in the field of methylation analysis comprises determining the probability of carry over contamination as specified above. According to a particular preferred embodiment, the determining the probability of carry over contamination comprises contacting of at least one identifier with a bisulfite reagent.

In a particular preferred embodiment, the testing of an experimental procedure comprises determining the efficiency of a reaction, of a step of said experimental procedure, of the complete experimental procedure, or combinations thereof. According to this embodiment, at least one or more identifiers substitute a biological sample (identifier sample). At least one identifier sample is subjected to the experimental procedure comprising at least one reaction or step, said step comprising at least one reaction. The efficiency is determined by considering the amount of at least one identifier before and after a reaction, before and after a step, or at the beginning and the end of the experimental procedure. The efficiency of a reaction is determined by the ratio of the amount of an identifier before the reaction and the amount of the said identifier or its derivative after the reaction. The efficiency of a procedure step is determined by the ratio of the amount of an identifier before the step and the amount of the said identifier or its derivative after the said step. The efficiency of a experimental procedure is determined by the ratio of the amount of an identifier at the beginning of the experimental procedure and the amount of the said identifier or its derivative at the end of the experimental procedure. Alternatively, the efficiency of a step of a experimental procedure is determined by multiplying the efficiencies of all reactions that are part of the said step with each other. Analogically, the efficiency of a complete experimental procedure is determined by either a) multiplying the efficiencies of all steps that are part of the experimental procedure with each other, or b) by multiplying the efficiencies of all reactions that are part of the experimental procedure with each other. A person skilled in the art is aware of further suitable algorithms. Those are herewith also preferred.

In a particular preferred embodiment, the testing of an experimental procedure in the field of methylation analysis comprises determining the efficiency of a reaction, of a step of said experimental procedure, of the complete experimental procedure, or combinations thereof. According to a particular preferred embodiment, the efficiency of a reaction, procedure step, or experimental procedure is determined wherein the said comprises contacting of at least one identifier with a bisulfite reagent.

In a particular preferred embodiment, the testing of an experimental procedure comprises optimizing the experimental procedure. According to this embodiment, at least one or more identifiers substitute a biological sample (identifier sample), and at least one identifier sample is applied to an experimental procedure. The experimental procedure is optimized by amending at least one step or reaction of the experimental procedure. This amendment is carried out according to different aims, for example, but not limited to, for a maximum amount of an identifier or its derivative at the end of the experimental procedure. Of course and also preferred, a person skilled in the art is aware of further aims. He is also aware of how to amend one or more reaction or one or more step to achieve a certain aim.

In a particular preferred embodiment, the testing of an experimental procedure in the field of methylation anaylsis comprises optimizing the experimental procedure as specified above. According to a particular preferred embodiment, the optimizing of the experimental procedure comprises contacting of at least one identifier with a bisulfite reagent.

In a particular preferred embodiment, the testing of an experimental procedure comprises assessing the success of a hybridization step. According to this embodiment, at least one or more identifiers substitute a biological sample (identifier sample), and at least one identifier sample is applied to an experimental procedure. This experimental procedure comprises an hybridization step which by itself may be characterized by different substeps like DNA preparation, prehybridization, hybridization, washing steps, or detection. Correspondingly any material, solution or substances may be used for this hybridization step as long as the hybridization is enabled of the processed applied identifier and the respective processed DNA derived from the biological sample(s), respectively.

In a particular preferred embodiment, the testing of an experimental procedure in the field of methylation anaylsis comprises assessing the success of a hybridization step of a experimental procedure as specified above. According to a particular preferred embodiment, the assessing the success of a hybridization step comprises contacting of at least one identifier with a bisulfite reagent.

In addition, particular aspects of the invention refer to controlling a process or method.

The following technical problems underlie the inventive embodiments for controlling the correctness of a process or method. Many laboratory routines require the parallel processing of samples, in particular a large number of samples. This is in particular the case for diagnostic, prognostic or screening purposes. Because of the large number of samples said processes or methods are highly accessible for errors. Such errors are for example but not limited to sample interchange or cross-contaminations. This error can occur on different levels of the analysis, for example but not limited to sample collection, sample preparation, DNA/RNA extraction, DNA/RNA modification, DNA/RNA amplification, or DNA/RNA characterization like PCR, Hybridization or sequencing. In addition, because of the large number of samples and a maybe limited number of applicable molecular identifiers, it might not be possible to assign a unique identifier to each sample. This is for example, but not limited to, the case, wherein the process or method comprises a real time PCR step. Only a limited number of dyes is detectable in real time PCR analysis, but up to 384 different sample can be measured in a single run.

The solution of the said problems is to use a small number of molecular identifiers e.g. 1-10 for spiking it/them into the samples in a defined order. Even if the number of samples is higher than the number of indentifiers an specific pattern of identifiers is expected at the end of the process. Every disorder of the samples is than easily recognizable by an unexpected order of the identifiers which are themselves identified according to a suitable method.

The particular advantage of this embodiment is that a lower number of molecular identifiers is needed to controll a process or method i.e. to exclude errors from a process or method. This results amongst others in lower handling efforts, costs, processing steps and a shorter analysis time.

In particular aspects, the method of the invention is a method for controlling the correctness of a process or method. Preferably said process or method is a high-throughput process or method. Preferably said process or method is a process or method for analysing DNA, genomic DNA or RNA. Preferably said process or method is a process or method for methylation analysis. The method of the invention comprises the following in arbitrary order:

A) The providing of a sample set of at least two or more biological samples which comprises DNA or RNA.

B) The assignment of one or more identifiers to the sample set, wherein preferably the identifiers are nucleic acids or at least in parts nucleic acids. Thereby the samples of the set become characterized by a pattern.

C) The execution of an experimental procedure, wherein all provided samples are analyzed. This analysis comprises for every sample the analysis of the provided sample DNA or RNA, the assigned at least one identifier, or both. Preferably this workflow enables detection or quantification of the methylation, the identifier, or both. Preferably the analysis of the sample DNA or RNA and the detection or quantification of the identifier are realized simultaneously.

In brief, in particular aspects, the method of the invention is a method for controlling the correctness of a process or method, comprising
providing a sample set of at least 2, 3, 4, 100, 200, 400, or 800 biological samples, wherein each sample comprises a nucleic acid;
applying at least one identifier to the sample set, wherein the applied at least one identifier does not interfere with subsequent analysis, and wherein the applied identifiers generate an identification pattern across the samples;
subjecting each sample to a detection or quantification reaction specific for the one or more applied identifiers;
subjecting each sample to analysis;
deducing the correctness of said process or method from the signals of the detected or quantified identifiers of the samples.

According to a preferred embodiment, at least one identifier is applied to each sample of the sample set.

According to a preferred embodiment, the method of the invention is a method for controlling the correctness of a process or method. Said method comprises the following:

(A) The providing of a sample set of 2, 3, 4, 10, 30, 60, 100, 200, 400, 800, 1000, 1500, 4000, 10000 or more samples. Thereby each samples comprises a nucleic acid to be analysed. Preferably said nucleic acid is genomic DNA or RNA. Of course and it is obvious to those skilled in the art said embodiment can also be applied to processes or methods, wherein proteins, peptides, metabolic compounds, hormons, lipids, cells, or combinations thereof are analyzed.

(B) The applying of at least one identifier to the sample set. Preferably only one identifier is applied to the sample set. Preferably only a single identifier is applied to each sample of the set. Preferably the total number of applied different identifiers is smaller than the total number of samples. Most preferably the total number of different identifiers is only 2, 3, 4, 5, 6, 7, 8, 9, 10.

The applied identifier or identifiers are characterized in that they enable subsequent analysis and they give rise to an identification pattern of the samples. Preferably, said pattern is a spatial or geometrical pattern. Preferably said pattern is any abstract order for example but not limited to numerical or alphabetical order. Preferably, every 2^{nd}, 3^{rd}, 4^{th}, 5^{th}, 6^{th}, 7^{th}, 8^{th}, 9^{th}, 10^{th} , etc. sample is encoded by the same identifier or group of identifiers. Preferably, the samples are placed in an hexagonal order and the same identifier or identifiers are applied to all samples of a row or a column. Preferably, the samples are placed in an radial order and the same identifier or identifiers are applied to all samples which have the same distance to a reference point for example the center. In a particular embodiment, the samples are encoded by two or more groups of identifiers. For example but not limited to, hexagonally orientated samples are encoded by a two groups of identifiers, wherein the first group encodes each row and the second groups encodes each column. Preferably each row, column or both are encoded by identifiers, wherein subgroups of the sample set may be formed, each sample of a subgroup be encoded by a specific combination of identifiers. Preferably one or more identifiers are applied to the sample set, whereby only every 2^{nd}, 3^{rd}, 4^{th}, 5^{th}, 6^{th}, 7^{th}, 8^{th}, 9^{th}, 10^{th}, etc. sample is encoded by one or more identifiers and all other samples of the set obtain no identifier. Thereby, correspondingly, a identification pattern is generated.

According to the invention for the method of controlling the correctness of a process or method, the applied one or more identifiers are identifier(s) as described above for the method of identifying at least one biological sample in the field of methylation analysis.

(C) The subjecting of each sample to a detection or quantification reaction specific for the one or more applied identifiers. Thereby the detection or quantification reaction is any kind of detection or quantification reaction. Preferably the detection or quantification reaction is a detection or quantification reaction as described above for the method of identifying at least one biological sample in the field of methylation analysis.

(D) The subjecting of each sample to analysis. Thereby each sample is analyzed by any kind of analysis, process or method. Preferably said analysis is carried out as described above for the method of identifying at least one biological sample in the field of methylation analysis. In particular each sample is analyzed with respect to the methylation of one or more CpG dinucleotides.

(E) The deducing the correctness of the process or method from the signals of the detected or quantified identifiers of the samples.

According to a preferred embodiment, the deducing the correctness of said process or method from the signals of the detected or quantified identifiers of the samples, comprises
determining the presence of an error-free process or method, wherein the said signals generate a pattern that is corresponds to the identification pattern as initially generated by applying the identifiers to the samples; or
determining the absence of an error-free process or method, wherein the said signals generate a pattern that does not correspond to the identification pattern as initially generated by applying the identifiers to the samples.

In a preferred embodiment, the correctness of said process or method is deduced from the signals of the detected or quantified identifiers of the samples. This assessment is achieved by comparing the pattern of the derived signals with the identification pattern generated by the applied identifier or indentifiers. In case the order or geometrical position of the samples relative to each other is not alternated during the process or method run, the pattern of the signals can be directly compared with the original identification pattern. In case the order or geometrical position of the samples relative to each other has be altered, the alterations have to be considered in the comparison with the original identification pattern. The presence of an accurate i.e. error-free process or method run is deduced, wherein the pattern of the signals is identical—no alteration of sample order or positions—, or resembles—alteration of sample order or positions—the originally identification pattern. On the other hand, the absence of an accurate i.e. error-free process or method run is deduced, wherein the pattern of the signals is not identical—no alteration of sample order or positions—, or resembles not—alteration of sample order or positions—the originally identification pattern.

According to a preferred embodiment, the process or method is a high-throughput process or method. Preferably it is a process or method in the field of methylation analysis and most preferably it is a high-throughput process or method in the field of methylation analysis.

According to the invention, the said embodiments for controlling the correctness of a process or method is applicable for quality ensurance or assessing the correctness of process runs for diagnostic, prognostic or screening purpusses. Amongst others, it is in particular suitable for the application in reference laboratories.

Kit.

The subject of the present invention is also a kit, comprising a container and one or more of the following:
at least one nucleic acid comprising at least one sequence polymorphic section;
at least one nucleic acid comprising at least one length polymorphic section;
at least one plasmid comprising at least one sequence polymorphic section;
at least one plasmid comprising at least one length polymorphic section;
at least one nucleic acid comprising at least one sequence polymorphic section and one length polymorphic section;
at least one oligonucleotide containing target-specific priming site and at least one sequence polymorphic section;
at least one oligonucleotide for amplifying at least one sequence polymorphic nucleic acid section, said oligonucleotide comprising DNA and/or DNA analogs like for example, but not limited to PNA, LNA, HNA, phosphothioate DNA, methylphosphonate DNA;
at least one oligonucleotide for amplifying at least one length polymorphic nucleic acid section, said oligonucleotide comprising DNA and/or DNA analogs like for example, but not limited to PNA, LNA, HNA, phosphothioate DNA, methylphosphonate DNA;
at least one nucleic acid for hybridization on at least one sequence polymorphic nucleic acid section;
at least one nucleic acid for hybridization on at least one length polymorphic nucleic acid section;
at least one antibody specific for one selected from the group comprising a protein, a peptide, a tag, a dye, a saccharide, a hormon, a lipid, a particle or combinations thereof;
at least one nucleic acid further comprising a protein, peptide, tag, dye, saccharide, hormon, lipid, nucleic acid, mass label, particle or combinations thereof; and
a description for carrying out the method of the invention. Preferably, a said kit further comprises a description for interpretation of results obtained by means of embodiments described herein.

A particular preferred kit comprises
at least one nucleic acid comprising at least one variant of a sequence polymorphism, at least one variant of a length polymorphism, or both, and
at least one oligonucleotide for amplifying at least one variant of a sequence polymorphism, at least one oligonucleotide for amplifying at least one variant of a length polymorphism, or both. A particular kit comprises further at least one nucleic acid for hybridization on at least one variant of a sequence polymorphism, at least one nucleic acid for hybridization on at least one variant of a length polymorphism, or both. Preferably, said oligonucleotides and/or nucleic acids comprises DNA and/or DNA analogs like for example, but not limited to PNA, LNA, HNA, phosphothioate DNA, methylphosphonate DNA.

Another particular kit comprises
at least one nucleic acid comprising at least one variant of a sequence polymorphism, at least one variant of a length polymorphism, or both, and
at least one nucleic acid for hybridization on at least one variant of a sequence polymorphism, at least one nucleic acid for hybridization on at least one variant of a length polymorphism, or both. A particular preferred kit comprises further at least one oligonucleotide for amplifying at least one variant of a sequence polymorphism, at least one oligonucleotide for amplifying at least one variant of a length polymorphism, or both. Preferably, said oligonucleotides and/or nucleic acids comprises DNA and/or DNA analogs like for example, but not limited to PNA, LNA, HNA, phosphothioate DNA, methylphosphonate DNA.

For the said particularly preferred kits, it is further preferred that the said at least one nucleic acid is one or more plasmids or is derived from one or more plasmids. Accordingly, preferably, the said oligonucleotides are able to amplify the at least one nucleic acid. Also preferably, the said nucleic acids are variants of a polymorphism, whereby each variant is a identifier.

Another particular preferred kit comprises a set of particles,
whereby each part of the set comprises at least one particle of at lest one size, and
whereby the one or more particles of different parts are of different sizes.

Another particular preferred kit comprises a set of dyes,
whereby each part of the set comprises at least one dye of at least one color, and
whereby the one or more dyes of different parts are of different colors.

Another particular preferred kit comprises a set of antibodies and corresponding epitopes,
whereby each part of the set comprises at least one epitope which is detected by at least one defined antibody, and
whereby the one or more epitopes of different parts are detected by different antibodies.

A particular preferred kit is a kit for identification of a biological sample,
wherein the sample comprises genomic DNA differentially methylated at least at one position. Preferably such a kit is used in the field of methylation analysis.

In particular such a kit is used for detection of sample interchange, crosscontamination, or both.

In particular such a kit is used for identifying a sample in a pooled sample set.

In particular such a kit is used for detection of an amplification inhibition.

In particular such a kit is used for determining the rate of DNA conversion.

In particular such a kit is used for normalization of a sample, calibration of a sample, or both.

In particular such a kit is used for identification of a carry over contamination.

In particular such a kit is used assessing the success of a hybridization step.

### USE OF A METHOD OR A KIT OF THE INVENTION

The methods and kits disclosed herein are preferably used for the analysis of at least one DNA methylation status, at least one DNA methylation level, or of at least one DNA methylation pattern. Of course also combinations of the said are preferred.

Preferably, the embodiments and kits described herein are used for at least one selected from the group comprising detection of sample interchange; detection of crosscontamination; identifying a sample in a pooled sample set; detection of amplification inhibition; determining the rate of DNA conversion; normalization of a sample; calibration of a sample; identification of carry over contamination, assessing the success of a hybridization step or combinations thereof.

Preferably, a method or kit according of the invention is used for at least one of the following with regard to a patient or individual: diagnosing a condition, prognosing a condition, predicting a treatment response, diagnosing a predisposition for a condition, diagnosing a progression of a condition, grading a condition, staging a condition, classification of a condition, characterization of a condition, or combinations thereof, wherein the condition is a healthy condition or an adverse event, the adverse event comprises at least one category selected from the group comprising: undesired drug interactions; cancer diseases, proliferative diseases or therewith associated diseases; CNS malfunctions; damage or disease; symptoms of aggression or behavioral disturbances; clinical; psychological and social consequences of brain damages; psychotic disturbances and personality disorders; dementia and/or associated syndromes; cardiovascular disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, of the muscles, of the connective tissue or of the bones; endocrine and/or metabolic malfunction, damage or disease; and headaches or sexual malfunction.

Moreover, a method or a kit of the invention are preferably used for distinguishing cell types or tissue, or for investigating cell differentiation.

All references cited herein are incorporated by reference to their entirety.

### DEFINITIONS

In particular aspects, the term “identifier” refers to, but is not limited to, a molecule which has unique chemical, physical or biological properties when compared with other molecules. It further refers to a molecule which is unambiguously assigned to a sample. According to the invention the term “identifier” refers to a molecule which is at least in parts a nucleic acid or a nucleic acid analog.

In particular aspects, the term “variants of polymorphism” or “polymorphism” refers to, but is not limited to, one of two or more alternate forms or alleles of nucleic acids or parts of nucleic acids that differ in nucleotide sequence or have variable numbers of nucleotides, in particular repeated nucleotides. According to the invention this refers preferably to the sequence (single nucleotide polymorphism, sequence polymorphism) or to the length (length polymorphism, deletion polymorphism).

In particular aspects, the term “methylation status” refers to, but is not limited to, the presence or absence of methylation of a single nucleotide in a single DNA molecule, said nucleotide being capable of being methylated.

In particular aspects, the term “methylation level” refers to, but is not limited to, the average methylation occupancy at a single nucleotide in a plurality of DNA molecules, said nucleotide being capable of being methylated.

In particular aspects, the term “methylation pattern” refers to, but is not limited to, the methylation status of a series of nucleotides located in cis on a single DNA molecule, said nucleotides being capable of being methylated.

In particular aspects, the term “remote sample” includes, but is not limited to, a sample having genomic DNA, wherein the sample is taken from a site (e.g., organ, tissue, body fluid, group of cells, cell, etc.) that is remote with respect to or that is distinct from the site of the cell, group of cells, tissue, or organ from which said genomic DNA originated.

In particular aspects, the term “crosscontamination” refers to, but is not limited to, an unintended addition of one or more components of a sample to another sample. Thereby the two samples are collected, processed, and/or analyzed at least in parts in parallel for example, but not limited to, as samples of the same sample set.

In particular aspects, the term “carry over contamination” refers to, but is not limited to, an unintended addition of one or more components of a sample to another sample. Thereby the two samples are collected, processed, and/or analyzed at least in parts one after the other, for example, but not limited to, as samples of different sample sets.

In particular aspects, the term “experimental procedure” refers to, but is not limited to, any combination of chemical, biological or physical reactions.

In particular aspects, the term “differentially methylated” refers to, but is not limited to, a state of a base of a nucleotide, the nucleotide being part of a nucleic acid, preferably of a DNA, in particular of a genomic DNA. Thereby said state is characterized in that the corresponding base is either methylated or unmethylated. Preferably the methylation or unmethylation is characteristic for an individual the corresponding sample is taken from, his state of health, the time at which the sample is taken, or combinations thereof.

### EXAMPLES

### Example 1

### Plant-Specific Fragments for the Identification of Sample Contamination and Sample Confusion During DNA Methylation Analysis

The Arabidopsis thaliana cellulose syntethase gene (SEQ ID NO: 1) At1g55850 has been checked for sequence homologies with the human genome using a BLAST search (http://www.ncbi.nlm.nih.gov/BLAST/Blast.cgi). Primers were designed which allow the amplification of fragments of different lengths by combining primer 1 with primer 2; primer 1 with primer 3; primer 1 with primer 4; primer 1 with primer 5; primer 1 with primer 6; primer 1 with primer 7; primer 1 with primer 8 and primer 1 with primer 9. The Combinations are summarized in Table 2.

TABLE 2
Designed plant-specific primer

| | | |
|---|---|---|
| Name | sequence | resulting fragment size |

| | | |
|---|---|---|
| primer 1 | 5′ccgctgcttacttgtcttcc3′ | |
| SEQ ID NO: 2 | | |
| primer 2 | 5′acagcttagccacctcctca3′ | 66 bp |
| SEQ ID NO: 3 | | |
| primer 3 | 5′ctccggtattcgtcccagt3′ | 122 bp |
| SEQ ID NO: 4 | | |
| primer 4 | 5′agcatcccactgtgaaaacc3′ | 167 bp |
| SEQ ID NO: 5 | | |
| primer 5 | 5′atggttccatggtttcttcg3′ | 196 bp |
| SEQ ID NO: 6 | | |
| primer 6 | 5′ttcccttctcttccatctacca3′ | 229 bp |
| SEQ ID NO: 7 | | |
| primer 7 | 5′ttttctcttgataaatacaccaacg3′ | 271 bp |
| SEQ ID NO: 8 | | |
| primer 8 | 5′cattgctccagccttgaagt3′ | 311 bp |
| SEQ ID NO: 9 | | |
| primer 9 | 5′ccaagtttagtatgattttcccaca3′ | 369 bp |
| SEQ ID NO: 10 | | |

In addition, 8 different domains were designed, which contain a cytosine-free oligonucleotide binding site (underlined in Table 3), a recognition site for Sca I (underlined and bold in Table 3), a cutting site for Swa I (bold in Table 3) and a unique identification site (twofold underlined in Table 3). pGem has a unique cutting site for Sca I. In that way the successful cloning of a fragment containing a single Sca I site can be recognized by gel analysis after a Sca I treatment. The Swa I cutting site is stable during bisulfite treatment and can be used to destroy bisulfite PCR contaminations.

TABLE 3
Domain primer

| | | fragment |
|---|---|---|
| Name | Sequence | size |
| domain-primer 1 | 5′GTGATGTGAGTTAATGATGGGccg | |
| SEQ ID NO: 11 | ctgcttacttgtcttcc3′ | |
| domain-primer 2 | 5′CCCTAACCTTAACATCTTCCAAGT | 66 bp |
| SEQ ID NO: 12 | ACTATTTAAATAACCATACTATACCA | |
| | AAATAATCACAGCTTAGCCACCTCCT | |
| | CA3′ | |
| domain-primer 3 | 5′AACCTTACTTTACCATACTCTAGT | 122 bp |
| SEQ ID NO: 13 | ACTATTTAAATAACCATACTATACCA | |
| | AAATAATCctccggtattcgtcccag | |
| | t3′ | |
| domain-primer 4 | 5′ATATAATCCAATAACCCCCAAGTA | 167 bp |
| SEQ ID NO: 14 | CTATTTAAATAACCATACTATACCAA | |
| | AATAATCagcatcccactgtgaaaa | |
| | cc3′ | |
| domain-primer 5 | 5′CACACCACCCAAAAACTAGTACTA | 196 bp |
| SEQ ID NO: 15 | TTTAAATAACCATACTATACCAAAAT | |
| | AATCatggttccatggtttcttcg3′ | |
| domain-primer 6 | 5′CACAATTACACATCCCAATAAACT | 229 bp |
| SEQ ID NO: 16 | TAGTACTATTTAAATAACCATACTAT | |
| | ACCAAAATAATCttcccttctcttcc | |
| | atctacca3′ | |
| domain-primer 7 | 5′CCCCACAATCAAACATACCATAGT | 271 bp |
| SEQ ID NO: 17 | ACTATTTAAATAACCATACTATACCA | |
| | AAATAATCttttctcttgataaatac | |
| | accaacg3′ | |
| domain-primer 8 | 5′TACAATCCAACTTAAAACCACTCA | 311 bp |
| SEQ ID NO: 18 | GTACTATTTAAATAACCATACTATAC | |
| | CAAAATAATCattgctccagccttg | |
| | aagt3′ | |
| domain-primer 9 | 5′CTCAACTCAATAAACCTTTACACA | 369 bp |
| SEQ ID NO: 19 | GTACTATTTAAATAACCATACTATAC | |
| | CAAAATAATCccaagtttagtatgat | |
| | tttcccaca3′ | |

Plant-specific fragments will be generated by using the following primer combinations: domain-primer 1+domain-primer 2; domain-primer 1+domain-primer 3; domain-primer 1+domain-primer 4; domain-primer 1+domain-primer 5; domain-primer 1+domain-primer 6; domain-primer 1+domain-primer 7; domain-primer 1+domain-primer 8 and domain-primer 1+domain-primer 9. Polymerase Chain Reaction will be performed in a total volume of 25 μl containing 10 ng Arabidopsis thaliana DNA, 1 U Hotstart Taq polymerase (Qiagen), 10 pmol of each forward and reverse primer, 1× PCR buffer (Qiagen) and 0.2 mmol/l of each dNTP (MBI Fermentas). Cycling will be done using a Mastercycler (Eppendorf) under the following conditions: 15 min at 95° C. and 15 cycles at 95° C. for 1 min, 60° C. for 45 s and 72° C. for 1:30 min and 30 cycles at 95° C. for 1 min, 72° C. for 1:30 min. 10 μl of the PCR mix will be loaded on a 2.5% agarose gel and fragments of the expected size will be cut out of the gel, purified using a gel-extraction kit (Qiagen) and used for TA-cloning (Promega). Successful cloning in a pGem vector will be verified by Sca I treatment and sequencing. In that way 8 different identifier plasmids will be created containing a sequence polymorphism and a length polymorphism.

2000 copies of two different plasmids will be added to each sample and deparafination, DNA extraction and bisulfite treatment will be performed together with the analyzed specimen. After bisulfite treatment bisulfit-independent primer will be used to analyze the identity of the samples by using the following primers: CF-primer 1 (SEQ ID NO: 20) GTGATGTGAGTTAATGATGGG and CF-primer 2 (SEQ ID NO: 21) AACCATACTATACCAAAATAATC. Polymerase Chain Reaction will be performed in a total volume of 25 μl containing 5 μl of bisulfite eluate, 1 U Hotstart Taq polymerase (Qiagen), 10 pmol of each forward and reverse primer, 1× PCR buffer (Qiagen) and 0.2 mmol/l of each dNTP (MBI Fermentas). Cycling will be done using a Mastercycler (Eppendorf) under the following conditions: 15 min at 95° C. and 45 cycles at 95° C. for 1 min, 55° C. for 45 s and 72° C. for 1:30 min. 5 μl PCR mix will be loaded on a 2.5% agarose gel and sample identity will be analyzed by fragment size (e.g. 66 bp and 311 bp) after gel electrophoresis. Sample contamination with PCR products can be identified by additional bands at the agarose gel (e.g. 66 bp; 122 bp and 311 bp). After bisulfite treatment the methylation of the sample will be analyzed in a duplex reaction using the following primers

| | |
|---|---|
| APC-F | |
| Cy5-GGAGAGAGAAGTAGTTGTGTAATT | (SEQ ID NO: 22) |
| and | |
| APC-R | |
| ACTACACCAATACAACCACATATC; | (SEQ ID NO: 23) |
| ID-primer 1 | |
| Cy5-tgtTttgattTtgTggTtga | (SEQ ID NO: 24) |
| and | |
| ID-primer 2 | |
| ccaacAcAttAAAaActctcc. | (SEQ ID NO: 25) |

Each capital letter represents a converted C; only complete converted DNA will be amplified. Polymerase Chain Reaction will be performed in a total volume of 25 μl containing 5 μl of bisulfite eluate, 1 U Hotstart Taq polymerase (Qiagen), 5 pmol of each primer, lx PCR buffer (Qiagen) and 0.3 mmol/l of each dNTP (MBI Fermentas). Cycling will be done using a Mastercycler (Eppendorf) under the following conditions: 15 min at 95° C. and 45 cycles at 95° C. for 1 min, 55° C. for 45 s and 72° C. for 1:30 min.

Bisulfite conversion and sample identification will be analyzed at a microarray using probes 1 to 8:

| | |
|---|---|
| probe 1 | |
| NH2-TGGAAGATGTTAAGGTTAGGG; | (SEQ ID NO: 26) |
| probe 2 | |
| NH2-AGAGTATGGTAAAGTAAGGTT; | (SEQ ID NO: 27) |
| probe 3 | |
| NH2-TGGGGGTTATTGGATTATAT; | (SEQ ID NO: 28) |
| probe 4 | |
| NH2-AGTTTTTGGGTGGTGTG; | (SEQ ID NO: 29) |
| probe 5 | |
| NH2-AAGTTTATTGGGATGTGTAATTGTG; | (SEQ ID NO: 30) |
| probe 6 | |
| NH2-ATGGTATGTTTGATTGTGGGG; | (SEQ ID NO: 31) |
| probe 7 | |
| NH2-GAGTGGTTTTAAGTTGGATTGTA; | (SEQ ID NO: 32) |
| probe 8 | |
| NH2-GAGTGGTTTTAAGTTGGATTGTA; | (SEQ ID NO: 33) |
| probe 9 | |
| NH2-GTGTAAAGGTTTATTGAGTTGAG. | (SEQ ID NO: 34) |

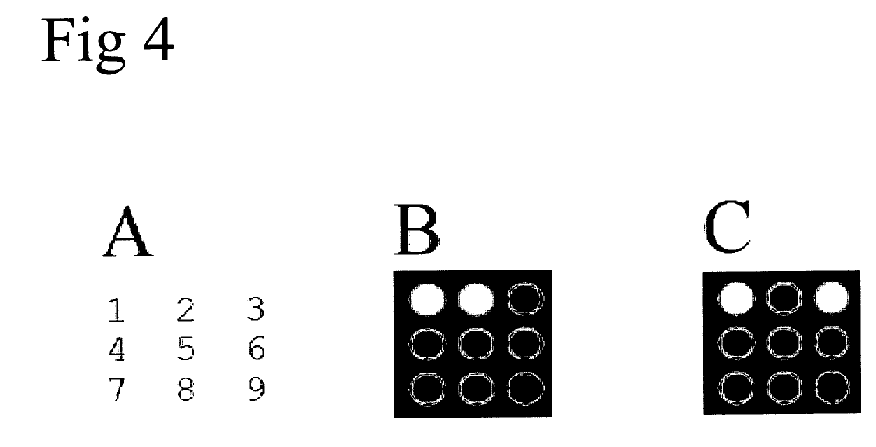

Hybridizations are carried out according to standard procedures. FIG. 4 shows schematic drawings of such a hybridization. In A the probe orientation at the array is shown. B and C show hybridizations of samples. In B two plasmids are used which are generated by domain-primer 1+domain-primer 2 and domain-primer 1+domain-primer 3, In C two plasmids are used which are generated by domain-primer 1+domain-primer 2 and domain-primer 1+domain-primer 4, Probes 1 to 8 hybridize with only one molecular identification plasmid. Probe 9 hybridizes with all plasmids.

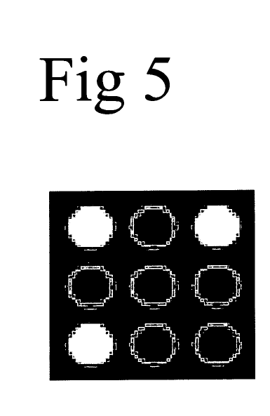

In FIG. 5 the detection of a contamination is visualized. FIG. 5 shows a schematic drawing of a hybridized microarray detecting a contamination of the sample. A combination of two plasmids was used.

The system is able to generate 28 different combinations of two different plasmids. In that way allowing both identification of sample and contamination.

Arabidopsis thaliana cellulose syntethase gene
(SEQ ID NO: 1) At1g55850
5′-ctcttatccctctcaccttctcacttggcaccgttgcagagagagaa
ccaaacatggtaaacaaagacgaccggattagaccggttcatgaagccga
cggtgaaccgctttttgagactaggagaagaaccggtagagtgattgcgt
accggtttttctcagcctcggttttcgtgtgtatctgtttgggtagtcac
acaatcttcccggtggaatccggtttggcgatttcccttctccgatagac
tctctcggagatacggaagcgaccttccgaggctcgacgtcttcgtttgc
acggcggatccggtgattgagccgccgttgttggtggtaaacacagtctt
atctgtgacggctcttgactacccaccggagaaactcgccgtttatctct
cagatgacggtggttctgagctgacgttctatgctctcacggaggcagct
gagtttgctaaaacttgggttcccttctgcaagaagttcaacgttgagcc
aacatctcccgctgcttacttgtcttccaaggcaaactgtcttgattctg
cggctgaggaggtggctaagctgtatagagaaatggcggcgaggattgaa
acggcggcgagactgggacgaataccggaggaggcgcgggtgaagtacgg
tgacgggttttcacagtgggatgctgacgctactcgaagaaaccatggaa
ccattcttcaagttttggtagatggaagagaagggaatacaatagcaata
ccaacgttggtgtatttatcaagagaaaagagacctcaacatcatcataa
cttcaaggctggagcaatgaacgcattgctgagggtttcttcgaaaatta
cttgtgggaaaatcatactaaacttggactgtgatatgtacgcaaacaac
tcaaagtcaacacgcgacgcgctctgcatcctcctcgatgagaaagaggg
aaaagagattgctttcgtgcagtttccgcagtgttttgacaatgttacaa
gaaatgatttgtatggaagcatgatgcgagtaggaattgatgtggaattt
cttggattggatggaaatggtggtccgttatacattggaactggatgctt
tcacagaagagatgtgatctgtggaagaaagtatggagaggaagaagaag
aagaagaatctgagagaattcacgaaaatttagagcctgagatgattaag
gctctcgcgagctgcacttatgaggaaaacactcaatggggaaaggagat
gggtgtgaaatatggttgcccggtagaggatgtaataactggtttgacga
ttcagtgtcgcggatggaaatcagcctacctgaacccggaaaagcaagca
tttctcggggtagcgccgaccaatttgcatcaaatgctagtgcagcagag
gagatggtcagagggagactttcagattatgctttcgaagtatagtccgg
tttggtatggaaaaggaaagatcagtttaggactgatacttggtcgagct
cgtggtttattccgtttggatacgtcactgttgcagctaccgcatatagc
ctagccgagttcttgtggtgcggagggacgttccgtggatggtggaacga
gcaaaggatgtggctttatagaagaacaagctcgtttcttttcggattta
tggacacgattaagaagctacttggagtttctgagtctgcgtttgtgatc
acagcaaaagtagcagaagaagaagcagcagagagatacaaggaagaggt
aatggagtttggagtggagtctcccatgtttctcgtcctcggaacactcg
gtatgctcaatctcttctgcttcgccgcagcggttgcgagacttgtttcc
ggagacggtggagatttgaaaacaatggggatgcaatttgtgataacagg
agtactagttgtcataaactggcctctgtataaaggtatgttgttgaggc
aagacaaaggaaagatgccaatgagcgttacagttaaatcagttgtttta
gctttatctgcctgtacctgtttagcgtttttgtaagattgattaacaac
agtcaaaaaagtaatcaaaataatgaccagcagttataatatgtaatttt
ct-3′

### Example 2

### Multiplex DNA Methylation Analysis by Usage of Domain Primers with Molecular Identifiers

Two samples are mixed with cytosine-free primers (200 pmol each) containing a molecular identification domain. Each sample is mixed with a different set of primers.

primer set 1:
set1F

(SEQ ID NO: 35)

5′TGATGGGAGAGTGAGTAGGA3′;
set1R

(SEQ ID NO: 36)

5′TGGAAGATGTTAAGGTTAGGGTCACTTCTAACTCTACCACTTA3′
primer set 2:
set2F

(SEQ ID NO: 37)

5′TGATGGGAGAGTGAGTAGGA3′;
set2R

(SEQ ID NO: 38)

5′AGAGTATGGTAAAGTAAGGTTTCACTTCTAACTCTACCACTTA3′

After bisulfite treatment with the EpiTect kit (Qiagen) samples are amplified as follows: Polymerase Chain Reaction is performed in a total volume of 25 μl containing 5 μl of bisulfite eluate, 1 U Hotstart Taq polymerase (Qiagen), 1× PCR buffer (Qiagen) and 0.2 mmol/l each dNTP (MBI Fermentas). Cycling is done using a Mastercycler (Eppendorf) under the following conditions: 15 min at 95° C. and 45 cycles at 95° C. for 1 min, 55° C. for 45 s and 72° C. for 1:30 min. Amplification mixtures are pooled and simultaneously hybridized on a microarray with the following capture probes and the following detection probes:

| | |
|---|---|
| capture probe 1 | |
| NH2-CCCTAACCTTAACATCTTCCA; | (SEQ ID NO: 39) |
| capture probe 2 | |
| NH2-AACCTTACTTTACCATACTCT; | (SEQ ID NO: 40) |

(capture probe 1 and capture probe 2 are able to hybridize onto the underlined part of the primers set 1R and set 2R, respectively.)

detection probe 1

(SEQ ID NO: 41)

Cy5-TAGAAAGTTTACGGTATTTTAAT (detection in case of
methylation);
detection probe 2

(SEQ ID NO: 42)

Cy3-TAGAAAGTTTATGGTATTTTAAT (detection in case of
unmethylation).

The methylation of each sample can be calculated by the Cy5/Cy3 signal ratio at the specific capture spot. In that way multiple samples can be analyzed in parallel.

### Example 3

### Performing a Methylation Detection Workflow Using a Molecular Identification Plasmid as Hybridization Control

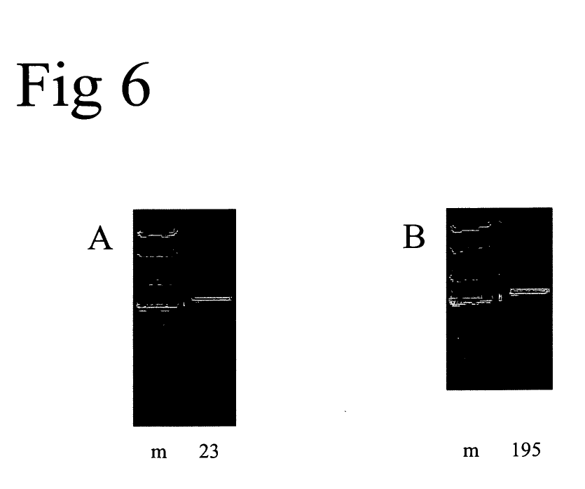

Two molecular identification plasmids (named 23 and 195) were generated. Therefore the two oligonucleotide pairs 23sens (SEQ ID NO: 43) AGTACTTGTATTTGAATTGTTTTTTTTGA/23anti (SEQ ID NO: 44) CAAAAAAAACAATTCAAATACAAGTACTA and 195sens (SEQ ID NO: 45) AGTACTGTATTTGGTTGGAGTGGGGA/195anti (SEQ ID NO: 46) CCCCACTCCAACCAAATACAGTACTA were cloned into pGem®-T vector, respectively. The said two oligonucleotide pairs are specific for oligonucleotides of an array tube (see below). Plasmids were isolated from transformed bacteria using a QIAprep Spin Miniprep kit (Qiagen). 500 ng plasmid DNA was linearized at 37° C. in 20 μl water containing 5 U of the restriction enzyme Bfu l and 1×NEB 4 buffer (both New England Biolabs). Reaction was stopped at 80° C. for 20 min. Clones were purified using the PCR-Purification kit (Qiagen). 100 fg plasmid DNA in 5 ng/μl poly-A solution (Roche) in a total volume of 27 μl were bisulfite treated using the EpiTect kit (Qiagen). Bisulfite DNA was amplified using the following primers MIDBis-1F (SEQ ID NO: 47) TGTGGAATTGTGAGtGGATA and MIDBis-1R (SEQ ID NO: 48) aCATaCTCCCaaCCaCCATa which are specific for sequences of the pGem-T vector. Therefore the following conditions were applied: total volume 25 μl; 1× QIA mPCR master mix MM (Qiagen) 0.2 pmol/l of each primer; Activation 95° C. for 15 min; Denaturation at 95° C. for 30 sec; Annealing at 61° C. for 45 sec; Extension at 72° C. for 1 min. Steps 2-4 were repeated 40 times. Finally an extension at 72° for 10 min was performed. PCRs were analyzed in an agarose gel and subsequently used for hybridization. FIG. 6 shows amplificates of the linearized bisulfite treated plasmid 23 and the linearized bisulfite treated plasmid 195, respectively.

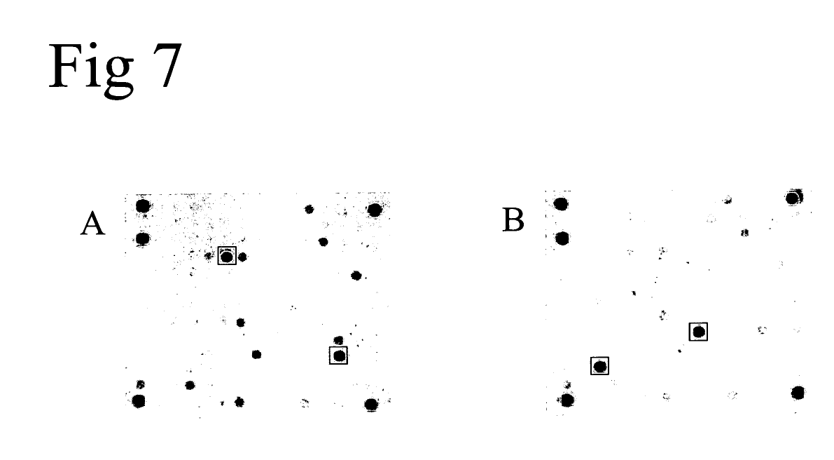

Hybridization was performed in array tubes provided by Clondiag Chip Technologies GmbH. These tubes contain a low density microarray with capture probes for methylation specific hybridization. The array tubes were prehybridized with 200 μl hybridization buffer (2× SSPE; 0.005% Triton) at 30° C. for 5 min. Each PCR product was diluted 1 to 100 in hybridization buffer and denatured for 10 min at 99° C. before hybridization. Hybridizations were performed for 1 h 10 min in a total volume of 100 μl at 35° C. The array tubes were washed three times with washing buffer (2×SSC) at 20° C. for 5 min. Subsequently, the array tubes were blocked with blocking solution (Pierce) and incubated with conjugate solution (Poly-Horseradish Peroxidase Streptavidin Conjugate in 2× SSPE/0.005% Triton buffer) for 30 min at 30° C. Afterwards, the array tubes were washed three times with washing buffer at 20° C. for 5 min. 100 μl True Blue substrate were added (Pierce) into each tube before the tubes were scanned in an ATS scanner (Clondiag Chip Technologies). The results are shown in FIG. 7. The amplificates of each of the two molecular identification plasmids hybridizes specifically two oligonucleotides of the array tube (dark spots marked by quadrats). Dark spots at the corner of the image show controll spots necessary for scanning the array tubes. Light grey spots represent unspecific hybridization.

### Example 4

### Controlling the Accurateness of a Real Time PCR Analysis

16 different samples are measured in triplicates in a real time PCR.

(A) The samples are numbered from 1 to 16, Four different identifiers are used to encode the samples. Thereby every 4^{th} sample encloses the same ndentifier:

| sample number: | spiked identifier |
|---|---|
| 1, 5, 9, 13 | identifier W |
| 2, 6, 10, 14 | identifier X |
| 3, 7, 11, 15 | identifier Y |
| 4, 8, 12, 16 | identifier Z |

The samples with spiked identifiers are subjected to real time PCR analysis, wherein a polymorphism of a sample DNA intrinsic site as well as the identity of the identifier is determined. According to the real time PCR analysis the 1^{st}, 2^{nd}, 3^{rd}, . . . 16^{th} samples comprise the following identifier:

| | |
|---|---|
| sample | identified identifier |

1^{st} replicate

| | |
|---|---|
| 2^{nd}, 5^{th}, 9^{th}, 13^{th} | identifier W |
| 1^{st}, 6^{th}, 10^{th}, 14^{th} | identifier X |
| 3^{rd}, 7^{th}, 11^{th}, 15^{th} | identifier Y |
| 4^{th}, 8^{th}, 12^{th}, 16^{th} | identifier Z |

2^{nd} replicate

| | |
|---|---|
| 2^{nd}, 5^{th}, 9^{th}, 13^{th} | identifier W |
| 1^{st}, 6^{th}, 10^{th}, 14^{th} | identifier X |
| 3^{rd}, 7^{th}, 11^{th}, 15^{th} | identifier Y |
| 4^{th}, 8^{th}, 12^{th}, 16^{th} | identifier Z |

3^{rd} replicate

| | |
|---|---|
| 2^{nd}, 5^{th}, 9^{th}, 13^{th} | identifier W |
| 1^{st}, 6^{th}, 10^{th}, 14^{th} | identifier X |
| 3^{rd}, 7^{th}, 11^{th}, 15^{th}, 16th | identifier Y |
| 4^{th}, 8^{th}, 12^{th}, 16^{th} | identifier Z |

From this it becomes obviousness that two errors occurred because of unexpected changes in the order of identified identifiers. A sample interchange between sample 1 and 2 took place before the samples were divided up into triplicates. In addition, it is obvious that sample 16 was crosscontaminated with either sample 3, 7, 11, 15 or combinations thereof during the 3^{rd} replicate run.

(B) Alternatively, the samples (numbered 1-16) were arranged in a certain order in a microtiter plate. Three different identifiers are used to encode the samples. Thereby every 3rd sample of the set encloses the same identifier:

| sample number: | spiked identifier |
|---|---|
| 1, 4, 7, 10, 13, 16 | identifier X |
| 2, 5, 8, 11, 14 | identifier Y |
| 3, 6, 9, 12, 15 | identifier Z |

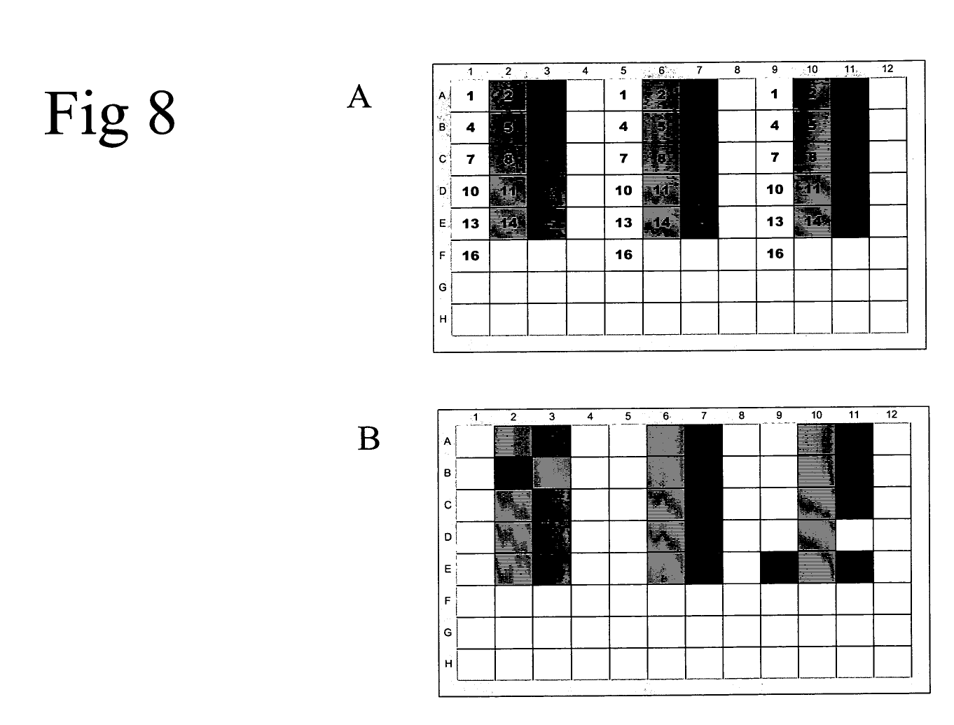

FIG. 8(a) gives a schematic overview over the so generated identifier pattern.

The samples with spiked identifiers are subjected to real time PCR analysis, wherein a polymorphism of a sample DNA intrinsic site as well as the identity of the identifier is determined. FIG. 8(b) shows a schematic overview of the results of the determination of the identifier identity, each result assigned to the position of the sample in the microtiter plate. Because the identifier pattern is in an unexpected order, an error is easily detected. It is obvious that samples 5 and 6 are switched in the first replicate (positions B2 and B3), and samples 12 and 13 in the third replicate (positions D11 and E9). Even if the identification of an individual sample is not 100% save (e.g. a switch of sample 1 and 3 would not be detected in this example) almost every process will be recognized.

I/we claim:

1. A method of identifying at least one biological sample in the field of methylation analysis, comprising
providing a sample set of at least one biological sample, wherein at least one sample comprises genomic DNA differentially methylated at least at one position;
applying at least one identifier for each sample, wherein the applied at least one identifier does not interfere with subsequent analysis;
subjecting each sample to a detection or quantification reaction specific for the one or more applied identifiers;
subjecting each sample to methylation analysis.

2. A method of claim 1, further comprising at least one of the following
contacting the DNA of each sample with a reagent or enzyme which differentiates between a methylated or an unmethylated position;
processing the sample set according to an experimental procedure.

3. A method of claim 1, comprising
providing a sample set of at least one biological sample, wherein at least one sample comprises genomic DNA differentially methylated at least at one position,
applying at least one identifier for each sample,
contacting the DNA of each sample with a reagent or enzyme which differentiates between a methylated or an unmethylated position,
detecting or quantifying the applied one or more identifiers for each sample,
subjecting each sample to methylation analysis.

4. A method of claim 3, wherein the detecting or quantifying the applied one or more identifiers for each sample and the methylation analysis of each sample are realized simultaneously.

5. A method of claim 1, wherein the identifier is at least in parts part of a larger molecule; part of an endogeneous molecule of the respective sample; part of an exogenous molecule added to the respective sample; a section of genomic DNA or total genomic DNA derived from a plant; a section of genomic DNA or total genomic DNA derived from a bacteria; a section of genomic DNA or total genomic DNA derived from a non-vertebrate; a section of genomic DNA or total genomic DNA derived from a vertebrate; a short tandem repeat; a variant of a deletion polymorphism; a variant of a single nucleotide polymorphism; a variant of a length polymorphism; an artificial nucleic acid; a circular nucleic acid; a circular DNA; a plasmid; a polynucleotide; an oligonucleotide; a PNA; a PNA-oligomer; a PNA-polymer; an artificial methylation; or combinations thereof.

6. A method of claim 1, wherein different identifiers are assigned to different sets of identifiers according to their respective biological, chemical, or physical properties.

7. A method of claim 6, characterized in that a representative of each of at least two sets of identifiers is comprised in a plasmid.

8. A method of claim 7, wherein the first set of identifiers comprises a sequence polymorphism and wherein the second set of identifiers comprises a length polymorphism.

9. A method of claim 1, wherein the identifier is a nucleic acid and additionally
forms no stable secondary structure;
comprises at least one oligonucleotide binding site covering converted cytosine positions;
is characterized by a similar base composition as the analyzed genomic DNA of the provided sample;
is a polymorphic sequence of about 5, about 10, about 15, about 20, about 25, about 30, about 35, about 40, about 50, about 75, about 100, or about 200 nucleotides;
has a content of cytosin-nucleotides and guanosin-nucleotides of about 15%, about 20%, about 30%, about 40%, about 50%, about 60%, about 70%, or about 80%; or combinations thereof.

10. A method of claim 1, wherein the identifier is a nucleic acid and additionally
forms no stable secondary structure;
comprises at least one cytosine-free, guanin-free or cytosine-free and guanin-free oligonucleotide binding site;
is characterized by a similar base composition as the analyzed genomic DNA of the provided sample;
is a polymorphic sequence of about 5, about 10, about 15, about 20, about 25, about 30, about 35, about 40, about 50, about 75, about 100, or about 200 nucleotides;
has a content of cytosin-nucleotides and guanosin-nucleotides of about 15%, about 20%, about 30%, about 40%, about 50%, about 60%, about 70%, or about 80%; or
combinations thereof.

11. A method of claim 1, wherein the identifier is a variant of a sequence polymorphism and additionally
comprises about 1, about 10, about 20, about 30, about 40, about 50, about 60, about 70, about 80, about 90, or about 100 variable nucleotide sites;
forms no stable secondary structure; or both.

12. A method of claim 1, wherein the identifier is a variant of a sequence polymorphism and additionally
comprises about 5, about 10, about 15, about 20, or about 25 variable nucleotide sites;
forms no stable secondary structure;
has a content of cytosin-nucleotides and guanosin-nucleotides of about 20%, about 30%, about 40%, about 50%, about 60%, about 70%, or about 80%; or
combinations thereof.

13. A method of claim 1, wherein the identifier is a variant of a length polymorphism and additionally
has a length difference of about 10, about 100, about 200, about 300, about 400, about 500, about 600, about 700, about 800, about 900, or about 1000 nucleotides compared to other used nucleic acid length polymorphic identifiers;
is of about 10, about 100, about 500, about 1.000, about 1.500, about 2.000, about 2.500, about 3,000, about 3,500, about 4,000, about 4,500, about 5,000, about 5,500, about 6,000, about 6,500, about 7,000, about 7,500, about 8,000, about 8,500, about 9.000, about 9,500, or about 10,000 nucleotides in length;
is derived from non-human DNA; or
combinations thereof.

14. A method of claim 1, wherein the identifier is a variant of a length polymorphism and additionally
is either of about 5, about 25, about 50, about 75, about 100, about 125, about 150, about 175, about 200, about 225, about 250, about 275, about 300, about 325, about 350, about 375, about 400, about 425, about 450, about 475, or about 500 nucleotides in length;
is derived from non-human DNA;
has a content of cytosin-nucleotides and guanosin-nucleotides of about 20%, about 30%, about 40%, about 50%, about 60%, about 70%, or about 80%; or
combinations thereof.

15. A method of claim 1, wherein the identifier comprises a tag selected from the group comprising dye, fluorescent dye, chemiluminescent dye, Cy5, Cy3, TAMRA, FAM, tag, epitope tag, peptide, polypeptide, protein, sacccharide, hormon, lipid, mass label, particle, gold particle, silver particle, platin particle, paraffin embedded code or combinations thereof.

16. A method of claim 2, wherein the reagent that differentiates between a methylated or an unmethylated position is a bisulfite reagent, wherein the methylated or unmethylated position is a cytosine position, or both.

17. A method of claim 2, wherein the experimental procedure comprises one or more of the following sample pooling, DNA isolation, DNA pooling, DNA concentration, DNA purification, bisulfite treatment, desulfonation, amplification.

18. A method of claim 1, wherein the detection or quantification reaction is carried out by one or more means selected from the group comprising: antibody, western blot analysis, chromatography, immunoassay, ELISA immunoassay, radioimmunoassay, FPLC, HPLC, UV light, light, spectrometer, MALDI-TOF, nucleic acid, DNA, PNA, oligonucleotide, PNA oligomer, amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, and Ms-SNuPE (Methylation-sensitive Single Nucleotide Primer Extension) method.

19. A method of claim 1, wherein the detection or quantification reaction comprises a nucleic acid, DNA, PNA, oligonucleotide, or PNA oligomer which
is at least of about 10, about 15, about 20, about 25, about 30, about 35, about 40, about 50, about 60, about 70, about 80, about 90, about 100, about 150 or about 200 nucleotides in length,
has a content of cytosin-nucleotides and guanosin-nucleotides of about 15%, about 20%, about 30%, about 40%, about 50%, about 60%, about 70%, about 80%, or about 85%;
has a melting temperature of about 37° C., about 45° C., about 50° C., about 55° C., about 60° C., about 65° C., about 70° C., about 75° C., about 80° C., about 85° C., about 90° C., about 95° C., or about 99° C.; or
combinations thereof.

20. A method of claim 1, wherein the detection or quantification reaction comprises a oligonucleotide which
is at least of about 5, about 10, about 15, about 20, about 25, about 30, about 35, about 40, about 50, about 60, about 70, about 80, or about 90 nucleotides in length;
has a content of cytosin-nucleotides and guanosin-nucleotides of about 5%, of about 10%, about 20%, about 30%, about 40%, about 50%, about 60%, about 70%, about 80%, about 90% or about 95%;
has a melting temperature of about 37° C., about 45° C., about 50° C., about 55° C., about 60° C., about 65° C., about 70° C., about 75° C., about 80° C., about 85° C., about 90° C., about 95° C., or about 99° C.; or
combinations thereof.

21. A method of claim 1, wherein the detection or quantification reaction comprises a oligonucleotide which
is at least of about 16, about 20, about 25, about 30, about 35, or about 40 nucleotides in length;
has a content of cytosin-nucleotides and guanosin-nucleotides of about 20%, about 30%, about 40%, about 50%, about 60%, about 70%, or about 80%; and
has a melting temperature of about 50° C., about 53° C., about 56° C., about 59° C., or about 62° C.

22. A method of claim 1, wherein the detection or quantification reaction comprises an oligonucleotide which comprises a gene-specific priming sequence and a sequence which hybridizes on a variant of a sequence polymorphism.

23. A method of claim 1, wherein the detection or quantification reaction comprises an oligonucleotide which comprises two domains,
wherein one domain comprises a target-specific priming sequence of about 10, about 15, about 20, about 25, about 30, about 35, or about 40 nucleotides, has a content of cytosin-nucleotides and guanosin-nucleotides of about 20%, about 30%, about 40%, about 50%, about 60%, about 70%, or about 80%, and has a domain melting temperature of about 50° C., about 52° C., about 54° C., about 56° C., about 58° C., about 60° C., or about 62° C.; and
wherein the other domain comprises a unique sequence of about 5, about 10, about 15, about 20, about 25, about 30, about 35, about 40, about 45, or about 50 nucleotides, is free of cytosines, guanin, or both, and has a content of cytosin-nucleotides and guanosin-nucleotides of about 20%, about 30%, about 40%, about 50%, about 60%, about 70%, or about 80%.

24. A method of claim 1, wherein methylation analysis comprises at least one selected from the group comprising detection of methylation status, detection of methylation level, detection of methylation pattern, detection of methylation pattern level, amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, and Ms-SNuPE (Methylation-sensitive Single Nucleotide Primer Extension) method.

25. A method of detection of sample interchange, crosscontamination, or both in the field of methylation analysis, comprising
providing a sample set of at least one biological sample, wherein at least one sample comprises genomic DNA differentially methylated at least at one position;
applying at least one identifier for each sample;
subjecting each sample with at least one identifier to a detection or quantification reaction that is specific for the at least one identifier; and
deducing the presence or absence of a sample interchange, of a crosscontamination, or both from the presence or absence of at least one identifier in a single sample.

26. A method of claim 25, wherein the step of deducing the presence or absence of a sample interchange, of a crosscontamination, or both further comprises
deducing the extent of a crosscontamination for a single sample from the absolute or relative amount of at least one identifier present in said single sample.

27. A method of identifying a sample in a pooled sample set in the field of methylation analysis, comprising
providing a pooled sample set of at least one biological sample, wherein at least one sample comprises genomic DNA differentially methylated at least at one position;
applying at least one identifier for each sample;
subjecting the sample set to a detection or quantification reaction that is specific for the at least one identifier of each sample; and
identifying a sample in the pooled sample set by detecting the respective applied at least one identifier.

28. A method of detection of an amplification inhibition in the field of methylation analysis, comprising
providing a sample set of at least one biological sample, wherein at least one sample comprises genomic DNA differentially methylated at least at one position;
applying at least one identifier for each sample;
subjecting each sample with at least one identifier to an amplification reaction that is specific for the at least one identifier; and
deducing a presence, absence or partial amplification inhibition from the presence, absence, or amount of the product of the identifier specific amplification reaction.

29. A method of normalization, calibration, or both in the field of methylation analysis, comprising
providing a sample set of at least one biological sample, wherein at least one sample comprises genomic DNA differentially methylated at least at one position;
applying at least one identifier for each sample by adding at least one identifier to each provided sample;
subjecting each sample with at least one identifier to a detection or quantification reaction; and
normalizing at least one sample, calibrating an experimental procedure, or both according to the detected or quantified one or more identifiers compared to the added total amount of one or more identifiers.

30. A method of identification of a carry over contamination in the field of methylation analysis, comprising
providing a sample set of at least one biological sample, wherein at least one sample comprises genomic DNA differentially methylated at least at one position;
applying at least one identifier for each sample;
subjecting each sample with at least one identifier to a detection or quantification reaction that is specific for at least one identifier;
deducing the presence of a sample carry over contamination from the presence of at least one identifier not applied for said sample, or deducing the absence of a sample contamination from the absence of identifiers not applied for said sample.

31. A method of assessing the success of a hybridization step in the field of methylation analysis, comprising
providing a sample set of at least one biological sample, wherein at least one sample comprises genomic DNA differentially methylated at least at one position;
applying at least one identifier;
subjecting each sample including the applied at least one identifier to a detection or quantification reaction that is specific for the said at least one identifier; wherein the detection or quantification reaction comprises a hybrization step,
assessing the success of the hybridization step wherein (a) the presence of a signal derived for the applied at least one identifier indicates the presence of a successful hybrization step, and wherein (b) the absence of signal derived for the applied at least one identifier indicates the presence of an unsuccessful hybrization step.

32. A method of any one of claims 25, 27, 28, 29, 30 or 31, further comprising contacting the DNA of each sample and the applied at least one identifier with a reagent or enzyme which differentiates between a methylated or an unmethylated position.

33. A method of determining the rate of DNA conversion in the field of methylation analysis, comprising
providing a sample set of at least one biological sample, wherein at least one sample comprises genomic DNA differentially methylated at least at one position;
applying at least one identifier for each sample, at least one of the applied identifiers comprises a cytosine that is not part of a CpG dinucleotide;
subjecting each sample with at least one identifier to at least one reaction that converts unmethylated cytosines to a base with a different base pairing behaviour than cytosine, while methylated cytosines remain unchanged;
subjecting the at least one identifier of each sample to at least one quantification reaction, wherein the total amount of identifier and the amount of converted identifier are detected; and
determining the rate of DNA conversion according to the amount of converted identifier compared to the total amount of identifier.

34. A method for testing an experimental procedure, comprising
applying at least one identifier instead of a biological sample to an experimental procedure;
subjecting the one or more identifiers to a detection or quantification reaction that is specific for the said one or more identifiers, and that is carried out before or after individual steps of the experimental procedure or subsequent to it.

35. A method of claim 34, wherein testing an experimental procedure comprises at least one of the following
determining the probability for a sample interchange, crosscontamination, or both;
determining the extent of a possible crosscontamination;
determining the probability of identifying a sample in a pooled sample set;
determining the probability of an amplification inhibition;
calibrating the experimental procedure;
determining the necessity of normalization;
determining the probability of carry over contamination;
determining the efficiency of a reaction, of a step of said experimental procedure, or of the complete experimental procedure;
optimizing the experimental procedure; and
determining the presence of a successful hybridization step or the presence of an unsuccessful hybridization step.

36. A method for controlling the correctness of a process or method, comprising providing a sample set of at least 2, 3, 4, 100, 200, 400, or 800 biological samples, wherein each sample comprises a nucleic acid;
applying at least one identifier to the sample set, wherein the applied at least one identifier does not interfere with subsequent analysis, and wherein the applied identifiers generate an identification pattern across the samples;
subjecting each sample to a detection or quantification reaction specific for the one or more applied identifiers;
subjecting each sample to analysis;
deducing the correctness of said process or method from the signals of the detected or quantified identifiers of the samples.

37. A method of claim 36, wherein at least one identifier is applied to each sample of the sample set.

38. A method of claim 36, wherein deducing the correctness of said process or method from the signals of the detected or quantified identifiers of the samples, comprises
determining the presence of an error-free process or method, wherein the said signals generate a pattern that is corresponds to the identification pattern as initially generated by applying the identifiers to the samples; or
determining the absence of an error-free process or method, wherein the said signals generate a pattern that does not correspond to the identification pattern as initially generated by applying the identifiers to the samples.

39. A method of claim 38, wherein the process or method is a high-throughput process or method.

40. A kit comprising a container and one or more of the following
at least one nucleic acid comprising at least one sequence polymorphic section;
at least one nucleic acid comprising at least one length polymorphic section;
at least one plasmid comprising at least one sequence polymorphic section;
at least one plasmid comprising at least one length polymorphic section;
at least one nucleic acid comprising at least one sequence polymorphic section and one length polymorphic section;
at least one oligonucleotide containing target-specific priming site and at least one sequence polymorphic section;
at least one oligonucleotide for amplifying at least one sequence polymorphic nucleic acid section;
at least one oligonucleotide for amplifying at least one length polymorphic nucleic acid section;
at least one nucleic acid for hybridization on at least one sequence polymorphic nucleic acid section;
at least one nucleic acid for hybridization on at least one length polymorphic nucleic acid section;
at least one antibody specific for one selected from the group comprising a protein, a peptide, a tag, a dye, a saccharide, a hormon, a lipid, a particle or combinations thereof;
at least one nucleic acid further comprising a protein, peptide, tag, dye, saccharide, hormon, lipid, nucleic acid, mass label, particle or combinations thereof;
a description for carrying out the method of the invention; and
a description for interpretation of results obtained by the method of the invention.

41. A kit of claim 40 comprising
at least one nucleic acid comprising at least one variant of a sequence polymorphism, at least one variant of a length polymorphism, or both; and
at least one oligonucleotide for amplifying at least one variant of a sequence polymorphism, at least one oligonucleotide for amplifying at least one variant of a length polymorphism, or both.

42. A kit of claim 41, further comprising at least one nucleic acid for hybridization on at least one variant of a sequence polymorphism, at least one nucleic acid for hybridization on at least one variant of a length polymorphism, or both.

43. A kit of claim 40 comprising
at least one nucleic acid comprising at least one variant of a sequence polymorphism, at least one variant of a length polymorphism, or both; and
at least one nucleic acid for hybridization on at least one variant of a sequence polymorphism, at least one nucleic acid for hybridization on at least one variant of a length polymorphism, or both.

44. A kit of claim 43, further comprising at least one oligonucleotide for amplifying at least one variant of a sequence polymorphism, at least one oligonucleotide for amplifying at least one variant of a length polymorphism, or both.

45. A kit of claim 41 or 43, whereby the said at least one nucleic acid is one or more plasmids or is derived from one or more plasmids.

46. A kit of claim 40 for identification of a biological sample,
wherein the sample comprises genomic DNA differentially methylated at least at one position.

47. A kit of claim 46 for detection of sample interchange, crosscontamination, or both.

48. A kit of claim 46 for identifying a sample in a pooled sample set.

49. A kit of claim 46 for detection of an amplification inhibition.

50. A kit of claim 46 for determining the rate of DNA conversion.

51. A kit of claim 46 for normalization of a sample, calibration of a sample, or both.

52. A kit of claim 46 for identification of a carry over contamination.

53. A kit of claim 46 for assessing the success of a hybridization step.

54. Use of a method or kit according to claim 1 for at least one selected from the group comprising detection of sample interchange; detection of crosscontamination; identifying a sample in a pooled sample set; detection of amplification inhibition; determining the rate of DNA conversion; normalization of a sample; calibration of a sample; identification of carry over contamination; controlling the success of a hybridization step or combinations thereof.

55. Use of a method or kit according to claim 54 for at least one of the following with regard to a patient or individual: diagnosing a condition, prognosing a condition, predicting a treatment response, diagnosing a predisposition for a condition, diagnosing a progression of a condition, grading a condition, staging a condition, classification of a condition, characterization of a condition, or combinations thereof, wherein the condition is a healthy condition or an adverse event, the adverse event comprises at least one category selected from the group comprising: undesired drug interactions; cancer diseases, proliferative diseases or therewith associated diseases; CNS malfunctions; damage or disease; symptoms of aggression or behavioral disturbances; clinical; psychological and social consequences of brain damages; psychotic disturbances and personality disorders; dementia and/or associated syndromes; cardiovascular disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, of the muscles, of the connective tissue or of the bones; endocrine and/or metabolic malfunction, damage or disease; and headaches or sexual malfunction.

56. Use of a method or kit according to claim 54 for distinguishing cell types or tissue, or for investigating cell differentiation.


SEQUENCE LISTING (48 sequences)

SEQ ID NO: 1 - DNA, 2309 bp, Homo Sapiens
ctcttatccctctcaccttctcacttggcaccgttgcagagagagaaccaaacatggtaaacaaagacgaccggattagaccggttcatgaagccgacggtgaaccgctttttgagactaggagaagaaccggtagagtgattgcgtaccggtttttctcagcctcggttttcgtgtgtatctgtttgatttggttctacagaattggtgagattggtgataaccgtaccgttttagatcgattaatctggtttgttatgtttattgtggagatttggttcggtttatattgggtagtcacacaatcttcccggtggaatccggtttggcgatttcccttctccgatagactctctcggagatacggaagcgaccttccgaggctcgacgtcttcgtttgcacggcggatccggtgattgagccgccgttgttggtggtaaacacagtcttatctgtgacggctcttgactacccaccggagaaactcgccgtttatctctcagatgacggtggttctgagctgacgttctatgctctcacggaggcagctgagtttgctaaaacttgggttcccttctgcaagaagttcaacgttgagccaacatctcccgctgcttacttgtcttccaaggcaaactgtcttgattctgcggctgaggaggtggctaagctgtatagagaaatggcggcgaggattgaaacggcggcgagactgggacgaataccggaggaggcgcgggtgaagtacggtgacgggttttcacagtgggatgctgacgctactcgaagaaaccatggaaccattcttcaagttttggtagatggaagagaagggaatacaatagcaataccaacgttggtgtatttatcaagagaaaagagacctcaacatcatcataacttcaaggctggagcaatgaacgcattgctgagggtttcttcgaaaattacttgtgggaaaatcatactaaacttggactgtgatatgtacgcaaacaactcaaagtcaacacgcgacgcgctctgcatcctcctcgatgagaaagagggaaaagagattgctttcgtgcagtttccgcagtgttttgacaatgttacaagaaatgatttgtatggaagcatgatgcgagtaggaattgatgtggaatttcttggattggatggaaatggtggtccgttatacattggaactggatgctttcacagaagagatgtgatctgtggaagaaagtatggagaggaagaagaagaagaagaatctgagagaattcacgaaaatttagagcctgagatgattaaggctctcgcgagctgcacttatgaggaaaacactcaatggggaaaggagatgggtgtgaaatatggttgcccggtagaggatgtaataactggtttgacgattcagtgtcgcggatggaaatcagcctacctgaacccggaaaagcaagcatttctcggggtagcgccgaccaatttgcatcaaatgctagtgcagcagaggagatggtcagagggagactttcagattatgctttcgaagtatagtccggtttggtatggaaaaggaaagatcagtttaggactgatacttggttactgttgctattgtctttgggctccatcttcactacctgtgctcatttactctgttttgacttctctctgtctcttcaaaggcattcctctgtttccaaaggtctcgagctcgtggtttattccgtttggatacgtcactgttgcagctaccgcatatagcctagccgagttcttgtggtgcggagggacgttccgtggatggtggaacgagcaaaggatgtggctttatagaagaacaagctcgtttcttttcggatttatggacacgattaagaagctacttggagtttctgagtctgcgtttgtgatcacagcaaaagtagcagaagaagaagcagcagagagatacaaggaagaggtaatggagtttggagtggagtctcccatgtttctcgtcctcggaacactcggtatgctcaatctcttctgcttcgccgcagcggttgcgagacttgtttccggagacggtggagatttgaaaacaatggggatgcaatttgtgataacaggagtactagttgtcataaactggcctctgtataaaggtatgttgttgaggcaagacaaaggaaagatgccaatgagcgttacagttaaatcagttgttttagctttatctgcctgtacctgtttagcgtttttgtaagattgattaacaacagtcaaaaaagtaatcaaaataatgaccagcagttataatatgtaattttct

SEQ ID NO: 2 - DNA, 20 bp, Homo Sapiens
ccgctgcttacttgtcttcc

SEQ ID NO: 3 - DNA, 20 bp, Homo Sapiens
acagcttagccacctcctca

SEQ ID NO: 4 - DNA, 19 bp, Homo Sapiens
ctccggtattcgtcccagt

SEQ ID NO: 5 - DNA, 20 bp, Homo Sapiens
agcatcccactgtgaaaacc

SEQ ID NO: 6 - DNA, 20 bp, Homo Sapiens
atggttccatggtttcttcg

SEQ ID NO: 7 - DNA, 22 bp, Homo Sapiens
ttcccttctcttccatctacca

SEQ ID NO: 8 - DNA, 25 bp, Homo Sapiens
ttttctcttgataaatacaccaacg

SEQ ID NO: 9 - DNA, 20 bp, Homo Sapiens
cattgctccagccttgaagt

SEQ ID NO: 10 - DNA, 25 bp, Homo Sapiens
ccaagtttagtatgattttcccaca

SEQ ID NO: 11 - DNA, 41 bp, Homo Sapiens
gtgatgtgagttaatgatgggccgctgcttacttgtcttcc

SEQ ID NO: 12 - DNA, 78 bp, Homo Sapiens
ccctaaccttaacatcttccaagtactatttaaataaccatactataccaaaataatcacagcttagccacctcctca

SEQ ID NO: 13 - DNA, 77 bp, Homo Sapiens
aaccttactttaccatactctagtactatttaaataaccatactataccaaaataatcctccggtattcgtcccagt

SEQ ID NO: 14 - DNA, 77 bp, Homo Sapiens
atataatccaataacccccaagtactatttaaataaccatactataccaaaataatcagcatcccactgtgaaaacc

SEQ ID NO: 15 - DNA, 74 bp, Homo Sapiens
cacaccacccaaaaactagtactatttaaataaccatactataccaaaataatcatggttccatggtttcttcg

SEQ ID NO: 16 - DNA, 84 bp, Homo Sapiens
cacaattacacatcccaataaacttagtactatttaaataaccatactataccaaaataatcttcccttctcttccatctacca

SEQ ID NO: 17 - DNA, 83 bp, Homo Sapiens
ccccacaatcaaacataccatagtactatttaaataaccatactataccaaaataatcttttctcttgataaatacaccaacg

SEQ ID NO: 18 - DNA, 80 bp, Homo Sapiens
tacaatccaacttaaaaccactcagtactatttaaataaccatactataccaaaataatccattgctccagccttgaagt

SEQ ID NO: 19 - DNA, 85 bp, Homo Sapiens
ctcaactcaataaacctttacacagtactatttaaataaccatactataccaaaataatcccaagtttagtatgattttcccaca

SEQ ID NO: 20 - DNA, 21 bp, Homo Sapiens
gtgatgtgagttaatgatggg

SEQ ID NO: 21 - DNA, 23 bp, Homo Sapiens
aaccatactataccaaaataatc

SEQ ID NO: 22 - DNA, 24 bp, Homo Sapiens
ggagagagaagtagttgtgtaatt

SEQ ID NO: 23 - DNA, 24 bp, Homo Sapiens
actacaccaatacaaccacatatc

SEQ ID NO: 24 - DNA, 20 bp, Homo Sapiens
tgttttgattttgtggttga

SEQ ID NO: 25 - DNA, 21 bp, Homo Sapiens
ccaacacattaaaaactctcc

SEQ ID NO: 26 - DNA, 21 bp, Homo Sapiens
tggaagatgttaaggttaggg

SEQ ID NO: 27 - DNA, 21 bp, Homo Sapiens
agagtatggtaaagtaaggtt

SEQ ID NO: 28 - DNA, 20 bp, Homo Sapiens
tgggggttattggattatat

SEQ ID NO: 29 - DNA, 17 bp, Homo Sapiens
agtttttgggtggtgtg

SEQ ID NO: 30 - DNA, 25 bp, Homo Sapiens
aagtttattgggatgtgtaattgtg

SEQ ID NO: 31 - DNA, 21 bp, Homo Sapiens
atggtatgtttgattgtgggg

SEQ ID NO: 32 - DNA, 23 bp, Homo Sapiens
gagtggttttaagttggattgta

SEQ ID NO: 33 - DNA, 23 bp, Homo Sapiens
gagtggttttaagttggattgta

SEQ ID NO: 34 - DNA, 23 bp, Homo Sapiens
gtgtaaaggtttattgagttgag

SEQ ID NO: 35 - DNA, 19 bp, Homo Sapiens
gatgggagagtgagtagga

SEQ ID NO: 36 - DNA, 43 bp, Homo Sapiens
tggaagatgttaaggttagggtcacttctaactctaccactta

SEQ ID NO: 37 - DNA, 20 bp, Homo Sapiens
tgatgggagagtgagtagga

SEQ ID NO: 38 - DNA, 43 bp, Homo Sapiens
agagtatggtaaagtaaggtttcacttctaactctaccactta

SEQ ID NO: 39 - DNA, 21 bp, Homo Sapiens
ccctaaccttaacatcttcca

SEQ ID NO: 40 - DNA, 21 bp, Homo Sapiens
aaccttactttaccatactct

SEQ ID NO: 41 - DNA, 23 bp, Homo Sapiens
tagaaagtttacggtattttaat

SEQ ID NO: 42 - DNA, 23 bp, Homo Sapiens
tagaaagtttatggtattttaat

SEQ ID NO: 43 - DNA, 29 bp, Homo Sapiens
agtacttgtatttgaattgttttttttga

SEQ ID NO: 44 - DNA, 29 bp, Homo Sapiens
caaaaaaaacaattcaaatacaagtacta

SEQ ID NO: 45 - DNA, 26 bp, Homo Sapiens
agtactgtatttggttggagtgggga

SEQ ID NO: 46 - DNA, 26 bp, Homo Sapiens
ccccactccaaccaaatacagtacta

SEQ ID NO: 47 - DNA, 20 bp, Homo Sapiens
tgtggaattgtgagtggata

SEQ ID NO: 48 - DNA, 20 bp, Homo Sapiens
acatactcccaaccaccata